(19) United States
(12) Patent Application Publication
(10) Pub. No.: US 2025/0064502 A1
(43) Pub. Date: Feb. 27, 2025

(54) Electrosurgical Devices, Methods of Use, and Methods of Manufacture

(71) Applicant: Stryker European Operations Limited, Cork (IE)

(72) Inventors: Micheal Burke, Cork (IE); Scott McFarland, Greenisland (IE); Paul Sheridan, Wexford (IE); Laura Constance Frey, Belfast (IE)

(21) Appl. No.: 18/719,132

(22) PCT Filed: Dec. 16, 2022
(86) PCT No.: PCT/IB2022/000771

§ 371 Date: Jun. 12, 2024

Related U.S. Application Data
(60) Provisional application No. 63/290,492, filed on Dec. 16, 2021.

(51) Int. Cl. A61B 18/12; A61B 18/00; A61B 18/14

(52) U.S. Cl. CPC A61B 18/1206; A61B 18/14; A61B 2018/00077; A61B 2018/00178; A61B 2018/00297; A61B 2018/00589; A61B 2018/00601; A61B 2018/00607

(57) ABSTRACT

In an example, an electrosurgical tool includes a housing. an electrosurgical electrode extending from a distal end of the housing, and an electrical cable extending from a proximal end of the housing. The electrical cable includes (I) a plug configured to electrically couple to an electrosurgical generator, (II) a proximal cable including a plurality of first conductors extending from the plug to a battery module, and (III) a distal cable comprising a plurality of second conductors extending from the battery module to the housing. The battery module includes: (a) a casing configured to receive a battery, and (b) a power driver circuit in an internal compartment of the casing. The power driver circuit includes: (i) a first set of contacts electrically coupled to the first conductors, (ii) a second set of contacts electrically coupled to the second conductors, and (iii) a third set of contacts electrically coupled to the battery.

### RELATED APPLICATIONS

The present application claims the benefit of priority of U.S. Provisional Application No. 63/290,492, filed Dec. 16, 2021, the entire contents of which is incorporated by reference in its entirety.

### FIELD

The present disclosure generally relates to electrosurgical devices and, more specifically, to electrosurgical devices and the methods for supplying electrosurgical energy and a direct current (DC) power during an electrosurgical procedure.

### BACKGROUND

Electrosurgery involves applying a radio frequency (RF) electric current (also referred to as electrosurgical energy) to biological tissue to cut, coagulate, or modify the biological tissue during an electrosurgical procedure. Specifically, an electrosurgical generator generates and provides the electric current to an active electrode, which applies the electric current (and, thus, electrical power) to the tissue. The electric current passes through the tissue and returns to the generator via a return electrode (also referred to as a “dispersive electrode”). As the electric current passes through the tissue, an impedance of the tissue converts a portion of the electric current into thermal energy (e.g., via the principles of resistive heating), which increases a temperature of the tissue and induces modifications to the tissue (e.g., cutting, coagulating, ablating, and/or sealing the tissue).

### SUMMARY

In an example, an electrosurgical tool includes a housing, an electrosurgical electrode extending from a distal end of the housing, and an electrical cable extending from a proximal end of the housing. The electrical cable includes (I) a plug configured to electrically couple to an electrosurgical generator, (II) a proximal cable including a plurality of first conductors extending from the plug to a battery module, and (III) a distal cable comprising a plurality of second conductors extending from the battery module to the housing. The battery module includes: (a) a casing configured to receive a battery, and (b) a power driver circuit in an internal compartment of the casing. The power driver circuit includes: (i) a first set of contacts electrically coupled to the first conductors, (ii) a second set of contacts electrically coupled to the second conductors, and (iii) a third set of contacts electrically coupled to the battery.

In another example, a method of operating an electrosurgical tool includes coupling an electrosurgical tool to an electrosurgical generator. The electrosurgical tool includes a housing, an electrosurgical electrode extending from a distal end of the housing, and an electrical cable extending from a proximal end of the housing. The electrical cable includes (I) a plug configured to electrically couple to an electrosurgical generator, (II) a proximal cable including a plurality of first conductors extending from the plug to a battery module, and (III) a distal cable comprising a plurality of second conductors extending from the battery module to the housing. The battery module includes: (a) a casing configured to receive a battery, and (b) a power driver circuit in an internal compartment of the casing. The power driver circuit includes: (i) a first set of contacts electrically coupled to the first conductors, (ii) a second set of contacts electrically coupled to the second conductors, and (iii) a third set of contacts electrically coupled to the battery.

The method also includes transmitting, by the proximal cable and the distal cable, the electrosurgical energy from the electrosurgical generator to the electrosurgical electrode. Additionally, the method includes performing, using the electrosurgical energy at the electrosurgical electrode, an electrosurgical operation. The method further includes transmitting a direct current (DC) power from the battery to a DC powered device.

In another example, a method of forming an electrosurgical tool includes forming a housing extending from a proximal end to a distal end, and coupling an electrosurgical electrode to the distal end of the housing. The electrosurgical electrode is configured to use electrosurgical energy to at least one of cut or coagulate tissue.

The method also includes forming an electrical cable configured to supply the electrosurgical energy from an electrosurgical generator. The electrical cable includes (I) a plug configured to electrically couple to an electrosurgical generator, (II) a proximal cable including a plurality of first conductors extending from the plug to a battery module, and (III) a distal cable comprising a plurality of second conductors extending from the battery module to the housing. The battery module includes: (a) a casing configured to receive a battery, and (b) a power driver circuit in an internal compartment of the casing. The power driver circuit includes: (i) a first set of contacts electrically coupled to the first conductors, (ii) a second set of contacts electrically coupled to the second conductors, and (iii) a third set of contacts electrically coupled to the battery.

In another example, an electrosurgical tool includes a housing extending from a proximal end to a distal end, and an electrosurgical electrode extending from the distal end of the housing. The electrosurgical electrode is configured to use electrosurgical energy to at least one of cut or coagulate tissue. The electrosurgical tool also includes an electrical cable extending from the proximal end of the housing. The electrical cable is configured to supply the electrosurgical energy from an electrosurgical generator.

The electrical cable includes a plug configured to electrically couple to the electrosurgical generator, and a battery module. The battery module includes: (i) a casing defining an internal compartment that is configured to receive a battery, and (ii) a battery printed circuit board (PCB) in the internal compartment of the casing, wherein the battery PCB comprises a first set of contacts that are configured to electrically couple a battery to the battery PCB. The electrical cable also includes a proximal cable extending from the plug to a battery module, a distal cable extending from the battery module to the housing, a plurality of electrosurgical energy (ES-energy) conductors extend an entire length of the electrical cable between the plug and the housing, and a plurality of direct current power (DC-power) conductors that extend from the battery PCB to the housing.

In another example, a method of operating an electrosurgical tool includes coupling an electrosurgical tool to an electrosurgical generator. The electrosurgical tool includes a housing, an electrosurgical electrode extending from a distal end of the housing, and an electrical cable extending from a proximal end of the housing. The electrical cable includes a plug configured to electrically couple to the electrosurgical generator, and a battery module. The battery module includes: (i) a casing defining an internal compartment that is configured to receive a battery, and (ii) a battery printed circuit board (PCB) in the internal compartment of the casing, wherein the battery PCB comprises a first set of contacts that are configured to electrically couple a battery to the battery PCB. The electrical cable also includes a proximal cable extending from the plug to a battery module, a distal cable extending from the battery module to the housing, a plurality of electrosurgical energy (ES-energy) conductors extend an entire length of the electrical cable between the plug and the housing, and a plurality of direct current power (DC-power) conductors that extend from the battery PCB to the housing.

The method also includes transmitting, by the proximal cable and the distal cable, the electrosurgical energy from the electrosurgical generator to the electrosurgical electrode. Additionally, the method includes performing, using the electrosurgical energy at the electrosurgical electrode, an electrosurgical operation. The method further includes transmitting a direct current (DC) power from the battery to a DC powered device.

In another example, a method of forming an electrosurgical tool includes forming a housing extending from a proximal end to a distal end, and coupling an electrosurgical electrode to the distal end of the housing. The electrosurgical electrode is configured to use electrosurgical energy to at least one of cut or coagulate tissue.

The method also includes forming an electrical cable configured to supply the electrosurgical energy from an electrosurgical generator. The electrical cable includes a plug configured to electrically couple to the electrosurgical generator, and a battery module. The battery module includes: (i) a casing defining an internal compartment that is configured to receive a battery, and (ii) a battery printed circuit board (PCB) in the internal compartment of the casing, wherein the battery PCB comprises a first set of contacts that are configured to electrically couple a battery to the battery PCB. The electrical cable also includes a proximal cable extending from the plug to a battery module, a distal cable extending from the battery module to the housing, a plurality of electrosurgical energy (ES-energy) conductors extend an entire length of the electrical cable between the plug and the housing, and a plurality of direct current power (DC-power) conductors that extend from the battery PCB to the housing.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying drawings, wherein:

FIG. 1 depicts a simplified block diagram of an electrosurgical system, according to an example.

FIG. 2 depicts a perspective view of an electrosurgical tool, according to another example.

FIG. 3 depicts a simplified block diagram of an electrical cable for the electrosurgical system of FIG. 1, according to an example.

FIG. 4A depicts a perspective view of a battery module in an open state, according to an example.

FIG. 4B depicts a first side view of the battery module of FIG. 4A in a closed state, according to an example.

FIG. 4C depicts a second side view of the battery module of FIG. 4A in the closed state, according to an example.

FIG. 4D depicts a side view of the battery module in the open state, according to an example.

FIG. 4E depicts a perspective view of a power driver circuit shown in FIG. 4A, according to an example.

FIG. 4F depicts a bottom view of the power driver circuit shown in FIG. 4E, according to an example.

FIG. 5 depicts a perspective view of the battery module of FIGS. 1-3 according to another example.

FIG. 6 depicts simplified block diagram of an electrical cable for the electrosurgical system of FIG. 1, according to another example.

FIG. 7A depicts an implementation of the electrical cable shown in FIG. 6, according to an example.

FIG. 7B depicts a cross-sectional view of the electrical cable shown in FIG. 7A, according to an example.

FIG. 8A depicts a top view of an internal compartment of a casing of a battery module in an open state, according to an example.

FIG. 8B depicts a bottom view of an exterior surface of the casing shown in FIG. 8A in the open state, according to an example.

FIG. 8C depicts a first side view of the casing shown in FIG. 8A in the open state, according to an example.

FIG. 8D depicts a side view of a distal end of the casing shown in FIG. 8A in the open state, according to an example.

FIG. 8E depicts a side view of a proximal end of the casing shown in FIG. 8A in the open state, according to an example.

FIG. 9 depicts a flowchart of a process of operating an electrosurgical tool, according to an example.

FIG. 10 depicts a flowchart of a process of forming an electrosurgical tool, according to an example.

FIG. 11 depicts a flowchart of a process of operating an electrosurgical tool, according to an example.

FIG. 12 depicts a flowchart of a process of forming an electrosurgical tool, according to an example.

### DETAILED DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As noted above, an electrosurgical tool can use electrical energy supplied by an electrosurgical generator to apply electrosurgical energy from an electrosurgical electrode to a tissue. In some instances, it can be beneficial to operate a direct current (DC) powered device before, during, or after an electrosurgical procedure. For example, the electrosurgical tool can include a light source that can be powered by a DC power to generate and emit light for illuminating an area of interest such as, for instance, a target tissue and/or a surgical site. As another example, the electrosurgical tool can include a camera that can be powered by a DC power to image the area of interest, and/or a sensor that can be powered by a DC power to sense a condition relating to the electrosurgical operation (and/or the electrosurgical tool).

One approach to providing the DC power for operating the DC powered device(s) is to incorporate a battery into a handle of the electrosurgical tool. However, this approach adds additional weight and size to the electrosurgical tool, which can make the electrosurgical tool less comfortable and/or more difficult for the user to handle.

Another approach to providing the DC power for operating the DC powered device(s) is to provide the battery in a receptacle that is external to the electrosurgical tool. This provides a benefit in that the battery is located outside the sterile environment around a patient in an operating room. Some of implementations of the external battery approach involve a standalone battery housing with a dedicated DC power cable that is separate from the power cable, which provides the electrosurgical energy from the electrosurgical generator to the electrosurgical tool. These implementations suffer from a drawback in that the external battery pack requires additional space on a counter, and the extra DC power cable can present challenges to cable management in an operating room.

Another implementation of the external battery involves incorporating the battery into a plug of the cable that couples the electrosurgical tool to the electrosurgical generator. While this approach provides a number of advantages over the approaches described above, this approach results in a relatively bulky plug that may inhibit access to other features on a surface of some electrosurgical generators. Additionally, for example, some electrosurgical generators may have a curved face and/or a relative deep socket where the plug couples to the electrosurgical generator, and these features may be incompatible with a relatively bulky plug.

The present application provides for an electrosurgical tool including a battery module that can address one or more of the challenges described above. In particular, the present application provides for a battery module that is incorporated into the electrical cable that couples the electrosurgical tool to the electrosurgical generator. In an example, the electrical cable includes (i) a plug configured to electrically couple to an electrosurgical generator, (ii) a proximal cable including a plurality of first conductors extending from the plug to a battery module, and (iii) a distal cable including a plurality of second conductors extending from the battery module to the housing. The plurality of first conductors can include a first quantity of conductors, the plurality of second conductors comprises a second quantity of conductors, and the first quantity is less than the second quantity.

The battery module can further include a casing defining an internal compartment that is configured to receive a battery, and a power driver circuit in the internal compartment of the casing. The power driver circuit can include: (i) a first set of contacts that are electrically coupled to the plurality of first conductors, (ii) a second set of contacts that are electrically coupled to the plurality of second conductors, and (iii) a third set of contacts that are electrically coupled to the battery.

In this arrangement, the plug can have a relatively smaller size than a plug that includes the battery. As a result, the electrosurgical tool can be compatible with relatively greater types of electrosurgical generators than some electrosurgical tools that incorporate the battery in the plug.

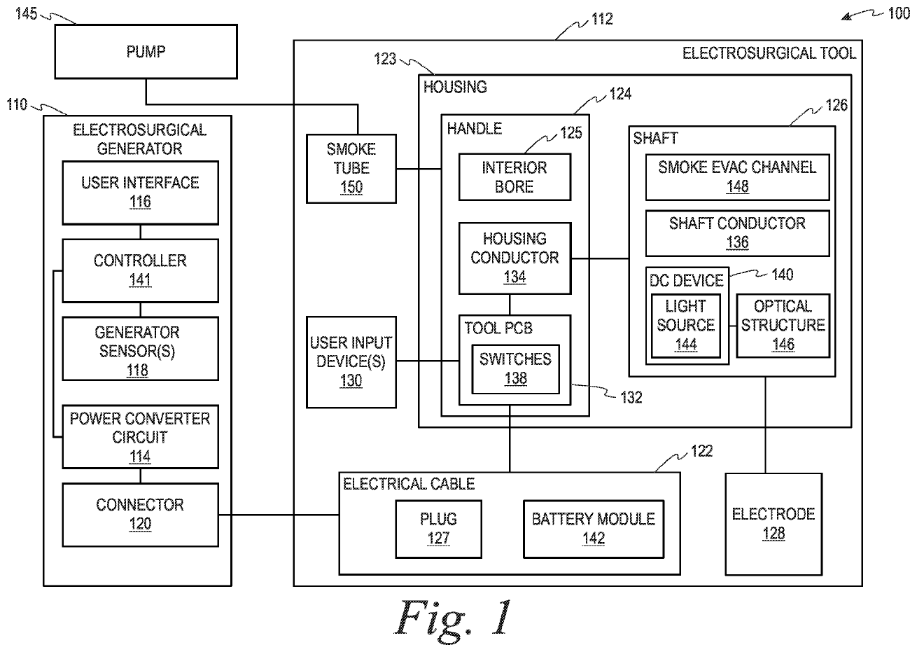

Referring now to FIG. 1, an electrosurgical system 100 is shown according to an example. As shown in FIG. 1, the electrosurgical system 100 includes an electrosurgical generator 110 and an electrosurgical tool 112. In general, the electrosurgical generator 110 can generate electrosurgical energy that is suitable for performing electrosurgery on a patient. For instance, the electrosurgical generator 110 can include a power converter circuit 114 that can convert a grid power to electrosurgical energy such as, for example, a radio frequency (RF) output power. As an example, the power converter circuit 114 can include one or more electrical components (e.g., one or more transformers) that can control a voltage, a current, and/or a frequency of the electrosurgical energy.

Within examples, the electrosurgical generator 110 can include a user interface 116 that can receive one or more inputs from a user and/or provide one or more outputs to the user. As examples, the user interface 116 can include one or more buttons, one or more switches, one or more dials, one or more keypads, one or more touchscreens, one or more display screens, one or more indicator lights, one or more speakers, and/or one or more haptic output devices.

In an example, the user interface 116 can be operable to select a mode of operation from among a plurality of modes of operation for the electrosurgical generator 110. As examples, the modes of operation can include a cutting mode, a coagulating mode, an ablating mode, and/or a sealing mode. Combinations of these waveforms can also be formed to create blended modes. In one implementation, the modes of operation can correspond to respective waveforms for the electrosurgical energy. As such, in this implementation, the electrosurgical generator 110 can generate the electrosurgical energy with a waveform selected from a plurality of waveforms based, at least in part, on the mode of operation selected using the user interface 116.

The electrosurgical generator 110 can also include one or more generator sensors 118 that can sense one or more conditions related to the electrosurgical energy and/or the target tissue. As examples, the generator sensor(s) 118 can include one or more current sensors, one or more voltage sensors, one or more temperature sensors, and/or one or more bioimpedance sensors. Within examples, the electrosurgical generator 110 can additionally or alternatively generate the electrosurgical energy with an amount of electrosurgical energy (e.g., an electrical power) and/or a waveform selected from among the plurality of waveforms based on one or more parameters related to the condition(s) sensed by the generator sensor(s) 118.

In one example, the electrosurgical energy can have a frequency that is greater than approximately 100 kilohertz (kHz) to reduce (or avoid) stimulating a muscle and/or a nerve near the target tissue. In another example, the electrosurgical energy can have a frequency that is between approximately 300 kHz and approximately 500 KHz.

In FIG. 1, the electrosurgical generator 110 also includes a connector 120 that can facilitate coupling the electrosurgical generator 110 to the electrosurgical tool 112. For example, the electrosurgical tool 112 can include an electrical cable 122 having a plug 127, which can be coupled to a socket of the connector 120 of the electrosurgical generator 110. In this arrangement, the electrosurgical generator 110 can supply the electrosurgical energy to the electrosurgical tool 112 via the coupling between the connector 120 of the electrosurgical generator 110 and the electrical cable 122 of the electrosurgical tool 112. The electrical cable 122 is described in further detail below.

The electrosurgical generator 110 can further include a controller 141 that can control operation of the electrosurgical generator 110. Within examples, the controller 141 can be implemented using hardware, software, and/or firmware. For instance, the controller 141 can include one or more processors and a non-transitory computer readable medium (e.g., volatile and/or non-volatile memory) that stores machine language instructions or other executable instructions. The instructions, when executed by the one or more processors, cause the electrosurgical generator 110 to carry out the various operations described herein. The controller 141, thus, can receive data and store the data in the memory as well. As shown in FIG. 1, the controller 141 can be communicatively coupled with the power converter circuit 114, the user interface 116, the generator sensor(s) 118, and/or the connector 120.

As shown in FIG. 1, the electrosurgical tool 112 can include a housing 123. The housing 123 can be an elongated structure in and/or on which components of the electrosurgical tool 112 can be disposed. In some examples, the housing 123 can be an integral, monolithic structure. In other examples the housing 123 can include a plurality of structures that are coupled to each other.

In FIG. 1, the housing 123 includes a handle 124 that defines an interior bore 125, a shaft 126 extending in a distal direction from the handle 124, and an electrosurgical electrode 128 extending in the distal direction from the shaft 126. In general, the handle 124 can be configured to facilitate a user gripping and manipulating the electrosurgical tool 112 while performing electrosurgery. For example, the handle 124 can have a shape and/or a size that can facilitate a user performing electrosurgery by manipulating the electrosurgical tool 112 using a single hand. In one implementation, the handle 124 can have a shape and/or a size that facilitates the user holding the electrosurgical tool 112 in a writing utensil gripping manner (e.g., the electrosurgical tool 112 can be an electrosurgical pencil).

Additionally, for example, the handle 124 and/or the shaft 126 can be constructed from one or more materials that are electrical insulators (e.g., a plastic material). This can facilitate insulating the user from the electrosurgical energy flowing through the electrosurgical tool 112 while performing the electrosurgery.

In some implementations, the shaft 126 can be coupled to the handle 124 in a fixed and non-moveable manner. This may simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts). In one example, the handle 124 and the shaft 126 can be formed as a single, monolithic structure such that the shaft 126 and the handle 124 are fixed and non-moveable relative to each other. In another example, the handle 124 and the shaft 126 can be fixedly coupled to each other by a welding coupling, an adhesive coupling, and/or another coupling that prevents movement between the handle 124 and the shaft 126.

In other implementations, the shaft 126 can be telescopically moveable relative to the handle 124. For example, the shaft 126 can be telescopically moveable in the interior bore 125 defined by the handle 124 to extend the shaft 126 in the distal direction and retract the shaft 126 in a proximal direction relative to the handle 124 (e.g., movable along a longitudinal axis of the electrosurgical tool 112). In some examples, the electrosurgical electrode 128 can be coupled to the shaft 126 and, thus, the electrosurgical electrode 128 can move together with the shaft 126 in an axial direction along the longitudinal axis relative to the handle 124. This can provide for adjusting a length of the electrosurgical tool 112, which can facilitate performing electrosurgery at a plurality of different depths within tissue (e.g., due to different anatomical shapes and/or sizes of patients) and/or at a plurality of different angles.

In some implementations, the electrosurgical electrode 128 can additionally or alternatively be rotatable about an axis of rotation that is parallel to the longitudinal axis of the electrosurgical tool 112. In some examples, the electrosurgical electrode 128 can be rotatable relative to the handle 124 and the shaft 126. In other examples, the electrosurgical electrode 128 can be rotationally fixed relative to the shaft 126 such that the shaft 126 and the electrosurgical electrode 128 are rotatable together relative to the handle 124. Rotating the electrosurgical electrode 128 relative to the handle 124 can facilitate adjusting an angle of the electrosurgical electrode 128 relative to one or more user input device(s) 130 of the electrosurgical tool 112. In this arrangement, a user can comfortably grip the handle 124 in a position in which their fingers can comfortably operate the user input device(s) 130 while the electrosurgical electrode 128 is set at a rotational position selected from among a plurality of rotational positions relative to the handle 124 based on, for example, a location, a size, and/or a shape of a surgical site in which the user is operating.

In one implementation, the electrosurgical electrode 128 can be rotatable by more than 360 degrees relative to the handle 124. This can improve an ease of use by allowing an operator to freely rotate the electrosurgical electrode 128 without limitation. However, in other implementations, the electrosurgical electrode 128 can be rotatable by less than or equal to 360 degrees (e.g., rotatable by 180 degrees or rotatable by 360 degrees). This may still allow an operator to achieve a desired rotational arrangement, but with the possibility that the operator may rotate in first direction, reach a stop limiting further rotation, and then rotate back in a second direction to achieve the desired rotational arrangement.

Although it can be beneficial to provide for rotation of the electrosurgical electrode 128 relative to the handle 124 and/or the shaft 126, the electrosurgical electrode 128 can be rotationally fixed relative to the handle 124 and the shaft 126 in some implementations. This may, for example, help to simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts).

The user input device(s) 130 can select between the modes of operation of the electrosurgical tool 112 and/or the electrosurgical generator 110. For instance, in one implementation, the user input device(s) 130 can be configured to select between a cutting mode of operation and a coagulation mode of operation. Responsive to actuation of the user input device(s) 130 of the electrosurgical tool 112, the electrosurgical tool 112 can (i) receive the electrosurgical energy with a level of power and/or a waveform corresponding to the mode of operation selected via the user input device(s) 130 and (ii) supply the electrosurgical energy to the electrosurgical electrode 128.

In FIG. 1, the electrosurgical tool 112 includes a plurality of electrical components that facilitate supplying the electrosurgical energy, which the electrosurgical tool 112 receives from the electrosurgical generator 110, to the electrosurgical electrode 128. For example. the electrosurgical tool 112 can include at least one electrical component selected from a group of electrical components including: a tool printed circuit board (tool PCB) 132 (e.g., a flexible printed circuit board), a housing conductor 134, and/or a shaft conductor 136 that can provide a circuit for conducting the electrosurgical energy from the electrical cable 122 to the electrosurgical electrode 128. One or more of the electrical components can be positioned in the interior bore 125 defined by the handle 124 and/or in the inner cavity defined by the shaft 126.

Within examples, the user input device(s) 130 can include one or more buttons on an exterior surface of the handle 124. Each button of the user input device(s) 130 can be operable to actuate a respective one of a plurality of switches 138 of the tool PCB 132. In general, the switches 138 and/or the tool PCB 132 are operable to control a supply of the electrosurgical energy from the electrosurgical generator 110 to the electrosurgical electrode 128. For instance, in one implementation, when each button is operated (e.g., depressed), the respective switch 138 associated with the button can be actuated to cause the tool PCB 132 to transmit a signal to the electrosurgical generator 110 and cause the electrosurgical generator 110 to responsively supply the electrosurgical energy with a level of power and/or a waveform corresponding to a mode of operation associated with the button. In another implementation, operating the button and thereby actuating the respective switch 138 associated with the button can close the switch 138 to complete a circuit to the electrosurgical generator 110 to cause the electrosurgical generator 110 to responsively supply the electrosurgical energy with a level of power and/or a waveform corresponding to a mode of operation associated with the button. In some examples of this implementation, the tool PCB 132 can be omitted.

In both example implementations, the electrosurgical energy supplied by the electrosurgical generator 110 can be supplied from (i) the electrical cable 122, the tool PCB 132, and/or the switches 138 to (ii) the electrosurgical electrode 128 by the housing conductor 134 and the shaft conductor 136. As such, as shown in FIG. 1, the tool PCB 132 can be coupled to the electrical cable 122, the housing conductor 134 can be coupled to the tool PCB 132 and the shaft conductor 136, and the shaft conductor 136 can be coupled to the electrosurgical electrode 128. In this arrangement, the housing conductor 134 can conduct the electrosurgical energy (supplied to the housing conductor 134 via the tool PCB 132) to the shaft conductor 136, and the shaft conductor 136 can conduct the electrosurgical energy to the electrosurgical electrode 128.

In general, the housing conductor 134 and the shaft conductor 136 can each include one or more electrically conductive elements that provide an electrically conductive bus for supplying the electrosurgical energy to the electrosurgical electrode 128. More particularly, the housing conductor 134 can include one or more electrically conductive elements of the handle 124 that can supply the electrosurgical energy to the shaft conductor 136, and the shaft conductor 136 can include one or more electrically conductive elements of the shaft 126 that can supply the electrical energy from the housing conductor 134 to the electrosurgical electrode 128. In implementations in which the shaft 126 is movable or rotatable relative to the handle 124, the housing conductor 134 can engage the shaft conductor 136 to maintain an electrical coupling between the housing conductor 134, the shaft conductor 136, and the electrosurgical electrode 128 while (i) the shaft 126 and/or the electrosurgical electrode 128 telescopically moves relative to the handle 124, and/or (ii) the electrosurgical electrode 128 rotates relative to the handle 124.

Although the electrosurgical tool 112 includes the user input device(s) 130 in FIG. 1, the user input device(s) 130 can be separate from the electrosurgical tool 112 in another example. For instance, the user input device(s) 130 can additionally or alternatively include one or more foot pedals that are actuatable to control operation of the electrosurgical tool 112 as described above. The foot pedal(s) can be communicatively coupled to the electrosurgical generator 110 to provide a signal responsive to actuation of the foot pedal(s).

As noted above, the electrosurgical electrode 128 can apply the electrosurgical energy to a target tissue to perform an electrosurgical operation (e.g., cutting, coagulating, ablating, and/or sealing the target tissue). Within examples, the electrosurgical electrode 128 can include an electrosurgical substrate formed from an electrically conductive material. As an example, the electrically conductive material can be stainless steel.

The electrosurgical substrate can extend in an axial direction from a proximal end of the electrosurgical electrode 128 to a distal end of the electrosurgical electrode 128. The proximal end of the electrosurgical electrode 128 can receive electrosurgical energy from the electrosurgical tool 112 (e.g., via the housing conductor 134 and the shaft conductor 136 as described above), and a distal working portion of the electrosurgical electrode 128 can apply the electrosurgical energy to the target tissue. In one implementation, the electrosurgical substrate can include a shank portion that extends from the proximal end of electrosurgical electrode 128 to the distal working portion of the electrosurgical electrode 128. The distal working portion can be configured to use the electrosurgical energy to at least one of cut or coagulate tissue in a monopolar electrosurgical operation.

In some examples, the distal working portion can define an electrosurgical blade. For instance, the electrosurgical blade can include (i) a first lateral surface, (ii) a second lateral surface opposite the first lateral surface, (iii) a first major surface extending between the first lateral surface and the second lateral surface on a first side of the electrosurgical blade, and (iv) a second major surface extending between the first lateral surface and the second lateral surface on a second side of the electrosurgical blade that is opposite the first side. The first lateral surface and the second lateral surface have surface areas that are relatively small compared to surface areas of the first major surface and the second major surface such that a thickness (e.g., a dimension between the first major surface and the second major surface) of the electrosurgical blade is relatively small as compared to a length (e.g., a dimension extending between the proximal end and the distal end of the electrosurgical electrode 128) and a width (e.g., a dimension between the first latera surface and the second lateral surface).

In some examples, the distal working portion of the electrosurgical electrode 128 can also include an outer layer of material covering at least a portion (or an entirety) of the electrosurgical substrate. For instance, the outer layer of material can be formed from at least one material selected from a group consisting of: a polymeric material, a fluorocarbon material (e.g., polytetrafluoroethylene (PTFE)), silicone, enamel, a ceramic material, and inorganic lubricant material (e.g., titanium nitride, zirconium nitride, titanium aluminum nitride, and nitron). The outer layer of material can help to, for example, inhibit eschar build-up and/or focus the electrosurgical energy to one or more portions of the electrosurgical electrode 128.

In some examples, the distal working portion of the electrosurgical electrode 128 can additionally include an intermediate layer between the electrosurgical substrate and the outer layer. The intermediate layer can be configured to provide thermal conductivity to help mitigate heating of the outer layer leading to a breakdown of the outer layer. The intermediate layer can also be configured to maintain the electrical conductivity of the electrosurgical substrate such that the intermediate layer does not degrade the transmission of the electrosurgical energy from the electrosurgical substrate to the target tissue.

The intermediate layer can be an anisotropic thermally conductive material, whereby the in-plane (e.g., parallel to the electrode surface) thermal conductivity substantially exceeds the out-of-plane (e.g., perpendicular to the electrode surface) thermal conductivity The anisotropic thermally conductive material having a coefficient of thermal expansion matched (or approximately 10% greater or approximately 10% lower) to the electrosurgical substrate and outer layer. As an example, this intermediate layer can include at least one material selected from a group consisting of: pyrolytic graphite/carbon, graphene, and Molybdenum disulfide.

Within examples, the electrosurgical tool 112 can additionally or alternatively include features that provide for evacuating surgical smoke from the distal end of the shaft 126 and/or the electrosurgical electrode 128 to a location external to the surgical site. Surgical smoke is a by-product of various surgical procedures. For example, during surgical procedures, surgical smoke may be generated as a by-product of electrosurgical units (ESU), lasers, electrocautery devices, ultrasonic devices, and/or other powered surgical instruments (e.g., bones saws and/or drills). In some instances, the surgical smoke may contain toxic gases and/or biological products that result from a destruction of tissue. Additionally, the surgical smoke may contain an unpleasant odor. For these and other reasons, many guidelines indicate that exposure of surgical personnel to surgical smoke should be reduced or minimized.

To reduce (or minimize) exposure to surgical smoke, a smoke evacuation system may be used during the surgical procedure. In general, the smoke evacuation system may include a suction pump 145 that can generate sufficient suction and/or vacuum pressure to draw the surgical smoke away from the surgical site. In some implementations, the smoke evacuation system may be coupled to an exhaust system (e.g., an in-wall exhaust system) that exhausts the surgical smoke out of an operating room. In other implementations, the smoke evacuation system may filter air containing the surgical smoke and return the air to the operating room. Within examples, the suction pump 145 and the electrosurgical generator 110 can be provided as separate devices or integrated in a single device (e.g., in a common housing).

As shown in FIG. 1, the shaft 126 can include a smoke evacuation channel 148 in the inner cavity of the shaft 126. The smoke evacuation channel 148 can also include a smoke inlet that can extend circumferentially around a center axis of a distal portion of the electrosurgical electrode 128. In this arrangement, the smoke inlet of the smoke evacuation channel can help to receive surgical smoke into the smoke evacuation channel 148 in all rotational alignments of the electrosurgical electrode 128 relative to the handle 124 and/or the electrosurgical tool 112 relative to the target tissue. However, in another example, the smoke evacuation channel 148 can include one or more smoke inlets that do not extend circumferentially around the electrosurgical electrode 128.

In an example, the smoke evacuation channel 148 of the shaft 126 defines a first portion of a smoke flow path, and the interior bore 125 of the handle 124 defines a second portion of a smoke flow path. In this arrangement, the surgical smoke can be received from the surgical site into the smoke evacuation channel 148 of the shaft 126, and flow proximally along the smoke evacuation channel 148 to the interior bore 125 of the handle 124. In the interior bore 125 of the handle 124, the smoke can further flow to a smoke tube 150 that is coupled to a proximal end of the handle 124 and configured to convey smoke from the handle 124 to the suction pump 145.

As shown in FIG. 1, the electrosurgical tool 112 includes at least one direct current (DC) device 140 and a battery module 142. In general, the DC device 140 is configured to use a DC power provided by the battery module 142 to perform a function in connection with the electrosurgical system 100. The DC device 140 can be disposed at least partially or entirely in the housing 123 and/or at least partially or entirely on an exterior surface of the housing 123. As examples, the DC device 140 can include at least one device selected from a group consisting of: one or more DC powered sensors, one or more cameras, one or more ultrasound transmitters, one or more light sources 144, one or more haptic devices, and one or more fluid pumps.

In examples that include a DC powered sensor, the DC power sensor can sense one or more operational conditions during an electrosurgical procedure. For instance, the DC powered sensor(s) can include at least one sensor selected from a group consisting of: (i) a temperature sensor, (ii) an electrochemical sensor, (iii) a force sensor, (iv) a mass loading sensor, (v) a dielectric sensor, (vi) a conductivity sensor, (vii) a metal detector sensor, (viii) a tracking sensor configured to sense at least one of: a location of the electrosurgical electrode and an orientation of the electrosurgical electrode, (ix) light sensor, and (x) a smoke detector sensor. Within examples, the DC powered sensor(s) transmit sensor signals to the controller 141 of the electrosurgical generator 110 to provide a basis for feedback control of the electrosurgical system 100 and improve the electrosurgical procedure.

In examples that include a camera, the camera can use the DC power provided by the battery module 142 to capture an image of an area of interest. For instance, the camera can be configured to have a field of view that is directed in a distal direction to capture an image of the electrosurgical electrode 128, a target tissue, and/or a surgical site. This can, among other things, help a user to visualize cutting and/or coagulating the target tissue.

In examples that include an ultrasound transmitter, the ultrasound transmitter can be used to detect a proximity to an electrically conductive object in a patient (e.g., one or more pacemaker leads). In one example, the ultrasound transmitter can be configured to, responsive to detecting a threshold proximity to the electrically conductive object, transmit a sensor signal to a controller (e.g., the controller 141 or the tool PCB 132) to cause the controller to cease and/or prevent a supply of the electrosurgical energy to the electrosurgical electrode 128. This can help to enhance safe operation of the electrosurgical system 100.

In examples that include the light source(s) 144, the light source(s) 144 can generate light that can be emitted by the electrosurgical tool 112 to illuminate an area of interest (e.g., a target tissue at the surgical site). In some implementations, the light source(s) 144 can be located at a distal end of the housing 123 and/or a distal end of the shaft 126 to directly provide light in a distal direction and illuminate a surgical distal of the electrosurgical electrode 128.

In other implementations, as shown in FIG. 1, the light source(s) 144 can be optically coupled to an optical structure 146, which is configured to receive the light emitted by the light source(s) 144 and transmit the light in a distal direction toward a surgical site to illuminate the surgical site while performing electrosurgery using the electrosurgical electrode 128. Although arranging the light source(s) 144 to directly illuminate a surgical field can help, for instance, to reduce a cost of manufacture, transmitting the light using the optical structure 146 can help to improve a quality of light transmitted from the electrosurgical tool 112 (e.g., by providing light with improved uniformity and/or reduced heat generation).

As examples, in implementations that include the optical structure 146, the optical structure 146 can include at least one optical structure selected from among a group consisting of an optical lens, a non-fiber optic optical waveguide, and an optical fiber. When the optical structure 146 includes the optical lens (e.g., a parabolic reflector lens, an aspheric lens, and/or a Fresnel lens), the optical structure 146 can help to direct the light emitted by the light source 144 in the distal direction and thereby improve a quality of the light illuminating the surgical site. The optical structure 146 can additionally or alternatively include the non-fiber optic optical waveguide and/or the optical fiber to transmit the light over relatively large distances in the shaft 126. For instance, the optical waveguide can transmit the light in the distal direction via total internal reflection. In such implementations, the optical waveguide can include a cladding and/or an air gap on an exterior surface of the optical waveguide to help facilitate total internal reflection. In some implementations, the non-fiber optic optical waveguide can be formed as a single, monolithic structure.

In some examples, the optical structure 146 can additionally or alternatively include other light shaping optical elements such as, for instance, a plurality of facets, one or more prisms, and/or one or more optical gratings. Although the optical structure 146 can help to improve a quality of the light directed to the surgical site, the electrosurgical tool 112 can omit the optical structure 146 and instead emit the light from the light source 144 directly to the surgical field without transmitting the light through the optical structure 146 in other examples.

In FIG. 1, the light source 144 can be coupled to the shaft 126. As such, the light source 144 can also move telescopically with the shaft 126 relative to the handle 124. However, in other examples, the light source 144 can be in the interior bore of the handle 124 and/or coupled to an exterior surface of the handle 124. As examples, the light source 144 can include one or more light emitting diodes (LEDs), organic light emitting diodes (OLEDs), optical fibers, non-fiber optic waveguides, and/or lenses. Additionally, for example, the light source 144 can include a light-emitting diode printed circuit board (LED PCB) having one or more light sources (e.g., LEDs). As described in further detail below, the LED PCB can include a PCB aperture, and one or more other components (e.g., the electrosurgical electrode 128) of the electrosurgical tool 112 can extend through the aperture.

The optical structure 146 can be at a distal end of the shaft 126. In some examples, the optical structure 146 can circumferentially surround the electrosurgical electrode 128 to emit the light distally around all sides of the electrosurgical electrode 128. This can help to mitigate shadows and provide greater uniformity of illumination in all rotational alignments of the shaft 126 relative to the housing 123 and/or the electrosurgical tool 112 relative to the target tissue. However, in other examples, the optical structure 146 can extend partially but not fully around the electrosurgical electrode 128.

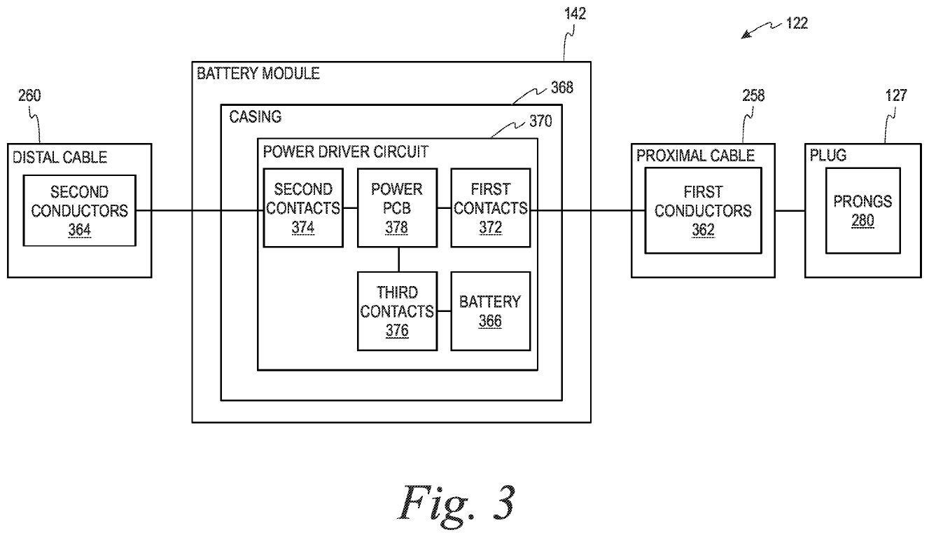

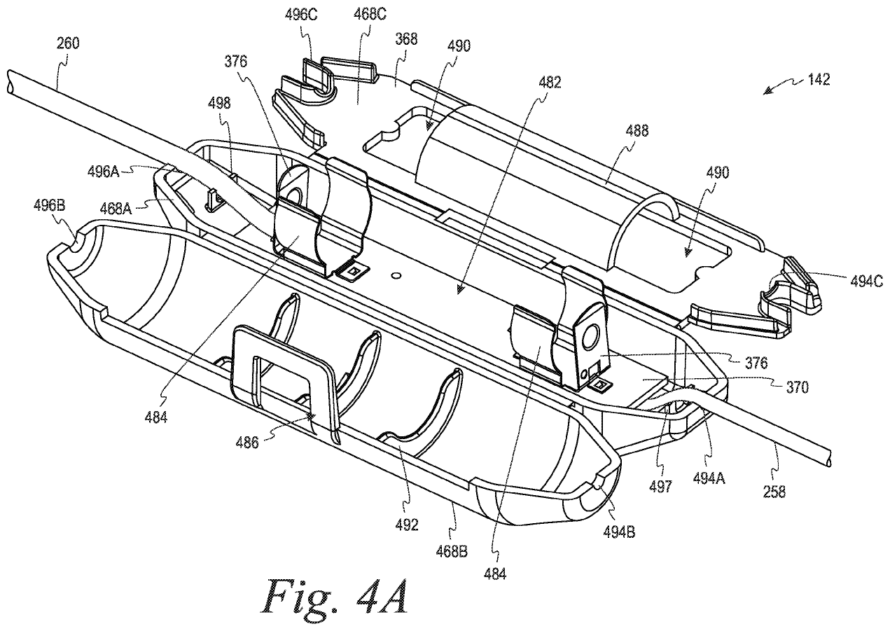

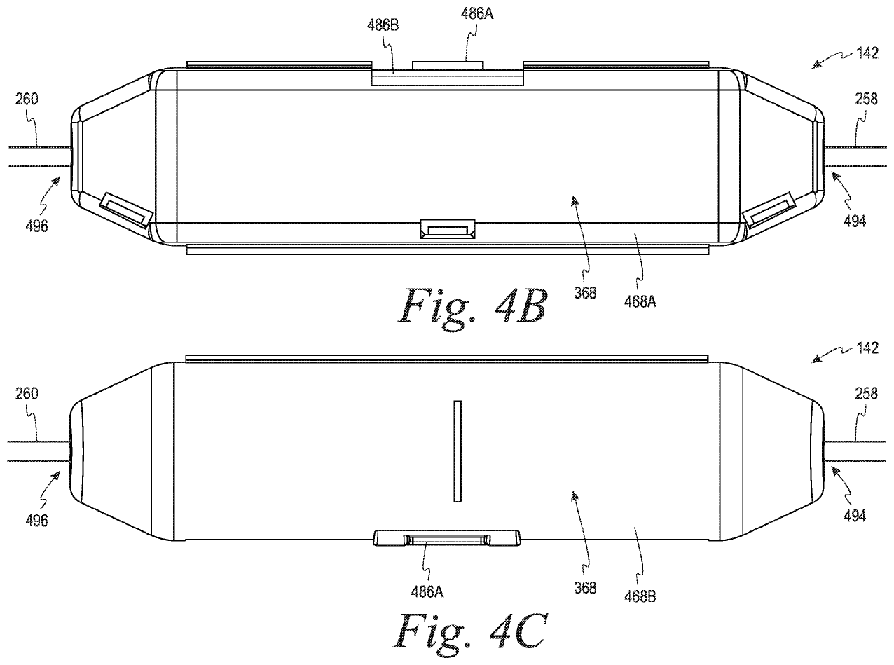

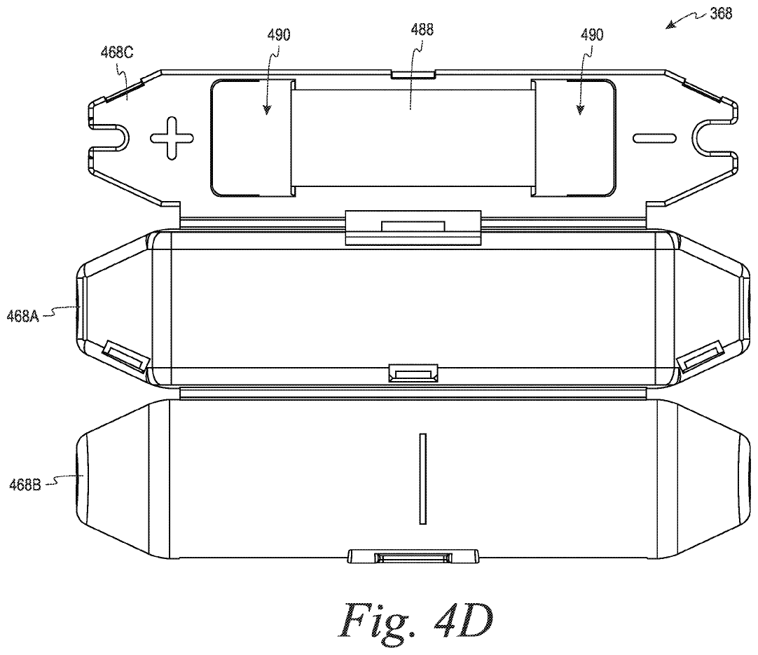

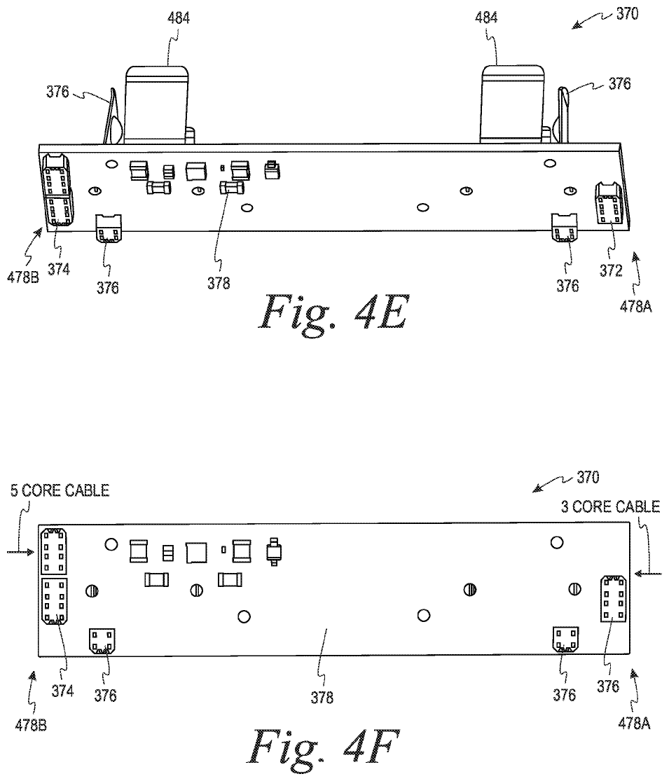

Within examples, the user input device(s) 130, the tool PCB 132, the switches 138, the housing conductor 134, the shaft conductor 136, the electrical cable 122, and/or the battery module 142 can supply the DC electrical power from the battery module 142 to the DC device 140. The electrical cable 122 and the battery module 142 are described in further detail below with respect to FIGS. 4A-4F.

The user input device(s) 130 can be actuated to operate the DC device(s) 140 (e.g., to cause the light source(s) 144 to emit light). In one example, the user input device(s) 130 can include a button that independently controls the DC device(s) 140 separate from the button(s) that control the electrosurgical operational modes of the electrosurgical tool 112. In another example, the user input device(s) 130 and the tool PCB 132 can be configured such that operation of the button(s) that control the electrosurgical operational mode simultaneously control operation of the DC devices 140 (e.g., the light source 144 can be automatically actuated to emit light when a button is operated to apply the electrosurgical energy at the electrosurgical electrode 128).

As shown in FIG. 1, responsive to operation of the user input device(s) 130 to actuate the DC device(s) 140, the battery module 142 can supply the electrical power (e.g., a DC voltage) to the DC device(s) 140 via the electrical cable 122, the tool PCB 132, the housing conductor 134, and/or the shaft conductor 136. In this implementation, one or more of the conductive elements of the housing conductor 134 can be configured to supply the electrical power from the battery module 142 to the DC device(s) 140 and/or return the electrical power from the DC device(s) 140 to the battery module 142. Accordingly, the housing conductor 134 can additionally or alternatively assist in providing electrical communication between the battery module 142 and the DC device(s) 140 as the shaft 126 and the light source 144 telescopically move and/or rotate relative to the handle 124.

Although the user input device(s) 130 on the handle 124 can be operated to control the operation of the DC device(s) 140 in the examples described above, the DC device(s) 140 can be additionally or alternatively operated by one or more user input device(s) on the electrosurgical generator 110 (e.g., via the user interface 116) and/or on the plug 127 of the electrical cable 122).

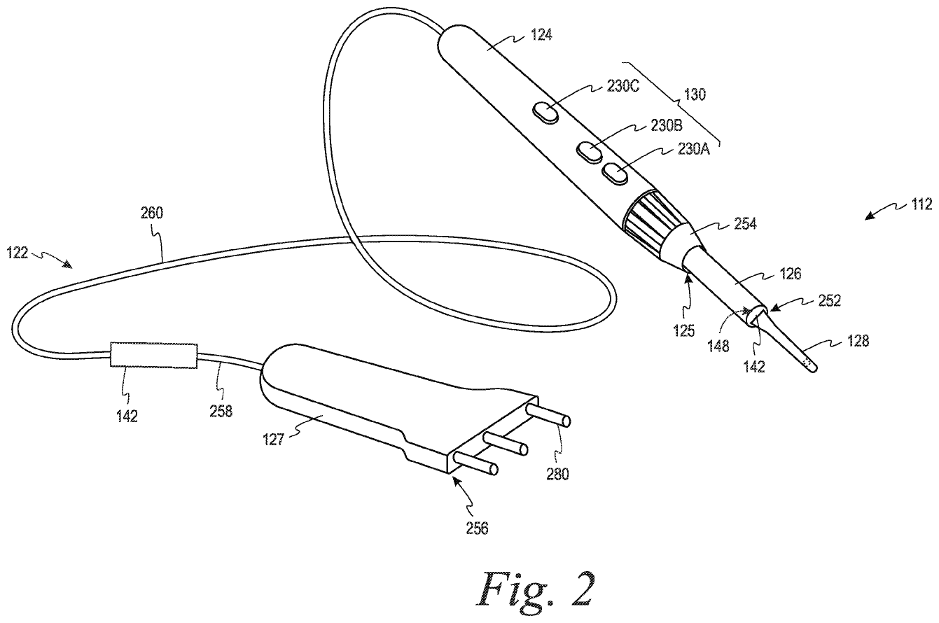

Referring now to FIG. 2, a perspective view of another implementation of the electrosurgical tool 112 is shown according to one example. In particular, FIG. 2 shows an implementation of the electrosurgical tool 112 that (i) includes illumination features and (ii) in which the shaft 126 is axially moveable and rotatable relative to the handle 124. As shown in FIG. 2, the electrosurgical tool 112 includes the housing 123 defining the interior bore 125, the shaft 126 telescopically moveable in the interior bore 125 of the housing 123, and the electrosurgical electrode 128 coupled to the shaft 126. However, as described above, the shaft 126 can be fixedly coupled to the handle 124 such that the shaft 126 is not moveable relative to the handle 124 in other examples.

Additionally, in FIG. 2, the optical structure 146 is at a distal end 252 of the shaft 126. In this arrangement, the optical structure 146 can telescopically move with the shaft 126 relative to the housing 123. In FIG. 2, the optical structure 146 extends around the electrosurgical electrode 128. This can help to emit the light in a relatively uniform manner by reducing (or preventing) shadows due to an orientation of the optical structure 146 and the electrosurgical electrode 128 relative to the surgical site. However, in other examples, the optical structure 146 may not extend entirely around the electrosurgical electrode 128 at the distal end 252 of the shaft 126, and/or the optical structure 146 can be at a different position on the shaft 126 and/or the housing 123. In still other examples, the electrosurgical tool 112 can omit the optical structure 146 and instead the light source 144 can be arranged at an exterior surface of the housing 123 to such that the light source 144 can directly illuminate the area of interest.

In some examples, the electrosurgical tool 112 can include a collar 254 at a proximal end of the housing 123. The collar 254 can be rotatable relative to the housing 123 to increase and/or decrease friction between an outer surface of the shaft 126 and an inner surface of the collar 254. In this way, the collar 254 to allow and/or inhibit axial telescopic movement of the shaft 126 relative to the housing 123.

Additionally, as shown in FIG. 2, the user input device(s) 130 include a first button 230A, a second button 230B, and a third button 230C on an exterior surface of the housing 123. In one implementation, the first button 230A can be actuated to operate the electrosurgical tool 112 in a cutting mode of operation, the second button 230B can be actuated to operate the electrosurgical tool 112 in a coagulation mode of operation, and the third button 230C can be actuated to operate the light source 144 (i.e., to cause the light source 144 to emit light or cease emitting light). As described above, the user input device(s) 130 can be configured differently in other examples. For instance, the electrosurgical tool 112 can be operable in a lesser quantity of modes of operation, a greater quantity of modes of operation, and/or different types of modes of operation in other examples (e.g., such as the example modes of operation described above). Additionally, for instance, the at least one user input device 130 can additionally or alternatively include the user interface 116 of the electrosurgical generator 110 and/or another external device (e.g., a footswitch) for operating the electrosurgical tool 112 in one or more modes of operation.

As shown in FIG. 2, the electrosurgical tool 112 includes the electrical cable 122. At a proximal end 256 of the electrical cable 122, the electrical cable 122 includes the plug 127 configured to couple to the connector 120 of the electrosurgical generator 110. A distal end of the electrical cable 122 is coupled to the tool PCB 132 in an interior cavity of the housing 123 (e.g., in the interior bore 125). In this arrangement, the electrical cable 122 extends proximally from the housing 123 to the plug 127 (e.g., from a proximal end of the housing 123 to the plug 127).

Additionally, as shown in FIG. 2, the electrical cable 122 includes the battery module 142 between the proximal end 256 of the electrical cable 122 and the distal end of the electrical cable 122. In this arrangement, the electrical cable 122 includes a proximal cable 258 that extends from the plug to the battery module 142 and a distal cable 260 that extends from the battery module 142 to the housing 123. By positioning the battery module 142 between the proximal cable 258 and the distal cable 260, the electrosurgical tool 112 can have a reduced size compared to an electrosurgical tool that incorporates the battery in the handle 124. Additionally, positioning the battery module 142 between the proximal cable 258 and the distal cable 260 can reduce a size and bulk of the plug 127 and better access to area(s) near the connector 120 on the electrosurgical generator 110 as compared to an electrosurgical tool 112 that incorporates the battery in the plug 127. Further, positioning the battery module 142 between the proximal cable 258 and the distal cable 260 can improve ease of replacing the battery as compared to the electrosurgical tools that incorporate the battery into the housing 123 or the plug 127.

In some examples, the proximal cable 258 has a first length, the distal cable 260 has a second length, and the second length is greater than the first length. This can help to position the battery module 142 nearer to the electrosurgical generator 110 than the handle 124 and, thus, farther away from a sterile environment (e.g., of a surgical site). As an example, the first length can be a length between approximately 10 cm and approximately 2 meters, and/or the second length can be between approximately 2 meters and approximately 5 meters.

In other examples, the second length can be less than the first length. This may be beneficial in an implementation in which the battery module 142 includes a user output device (e.g., a display and/or an indicator light) that provides information to a user during a procedure. For instance, when the second length is less than the first length, the battery module 142 can be closer to the operator, which may make it easier to receive information from the user output device (e.g., as compared to an alternative implementation in which the second length is greater than the first length and the battery module 142 is farther from the operator).

Referring now to FIG. 3, a simplified block diagram of the electrical cable 122 is shown according to an example. As shown in FIG. 3, the electrical cable 122 includes the plug 127 that is configured to electrically couple to the electrosurgical generator 110, the proximal cable 258 including a plurality of first conductors 362 extending from the plug 127 to the battery module 142, and the distal cable 260 including a plurality of second conductors 364 extending from the battery module 142 to the housing 123. In general, the first conductors 362 of the proximal cable 258 can supply the electrosurgical energy from the electrosurgical generator 110 to the battery module 142, whereas the second conductors 364 can supply from the battery module 142 both (i) the electrosurgical energy toward the electrosurgical electrode 128 and (ii) the DC power to the DC device 140.

For example, the first conductors 362 can include a first quantity of conductors, the second conductors 364 include a second quantity of conductors, and the first quantity is less than the second quantity. In this arrangement, the first conductors 362 and a first subset of the second conductors 364 are configured to supply the electrosurgical energy from the electrosurgical generator 110 toward the electrosurgical electrode 128, and a second subset of the second conductors 364 are configured to supply electrical power from a battery 366 of the battery module 142 to the DC device 140.

In one implementation, the first quantity of conductors is three and the second quantity of conductors is five. In this implementation, the first subset of the second conductors 364 includes three conductors for the electrosurgical energy, and the second subset of the second conductors 364 includes two conductors for the DC power. In an example, the three conductors 364 of the first subset can include a cut conductor for signaling the electrosurgical generator 110 to provide the electrosurgical energy for a cut operation, a coagulation conductor for signaling the electrosurgical generator 110 to provide the electrosurgical energy for a coagulation operation, and a power transmission conductor for supplying the electrosurgery energy from the electrosurgical generator 110 to the electrosurgical electrode 128. However, in other examples, the first conductors 362 and the second conductors 364 can include different quantities of conductors. For instance, the second quantity of the second conductors 364 can equal X+2Y, where X is the first quantity of the first conductors 362 and Y is a quantity of DC devices 140. As an example, in an implementation in which there three first conductors 362 and two DC devices 140, the second quantity of conductors can be seven.

As shown in FIG. 3, the battery module 142 includes a casing 368. The casing 368 defines an internal compartment that is configured to receive the battery 366. In some examples, the battery 366 can include a single battery. In other examples, the battery 366 can include a plurality of batteries. In some implementations, the casing 368 can completely enclose the battery 366. This can help to mitigate foreign matter (e.g., liquids) from contacting and affecting the operation of the battery 366. In other implementations, the casing 368 can retain the battery 366 without fully enclosing the battery 366.

In some examples, the battery 366 can be insertable and removable from the casing 368 (e.g., to facilitate inserting the battery 366 prior to first use of the electrosurgical tool 112 and/or to replace an expired battery with a new battery). In such examples, the casing 368 can be actuated between (i) a closed state in which the casing 368 inhibits or prevents access to the battery 366 in the internal compartment, and (ii) an open state in which the casing 368 permits access to the battery 366 in the internal compartment. In other examples, the battery 366 can be non-replaceable and the casing 368 can be configured to only inhibit access to the battery 366 in the internal compartment. For instance, the casing 368 can be configured such that the casting can be actuated from the open state to a closed state (e.g., during a manufacturing process and/or prior to first use of the electrosurgical tool 112), but cannot be returned from the closed state to the open state.

The battery module 142 can also include a power driver circuit 370 in the internal compartment of the casing 368. The power driver circuit 370 can include (i) a first set of contacts 372 that are electrically coupled to the first conductors 362, (ii) a second set of contacts 374 that are electrically coupled to the second conductors 364, and (iii) a third set of contacts 376 that are electrically coupled to the battery 366. The power driver circuit 370 can include one or more electrical components that can operate to control one or more functions related to providing the electrosurgical energy from the electrosurgical generator 110 to the electrosurgical electrode 128 (and/or the tool PCB 132), and/or providing the DC power from the battery 366 to the DC device 140. For instance, the power driver circuit 370 can include one or more electrical components that can provide for reverse polarity protection, surge protection, electromagnetic interference (EMI) protection, overcharging protection, over-discharging protection, and/or over-drain protection in connection with the battery 366. The power driver circuit 370 can additionally or alternatively provide a battery management system that can monitor a state of the battery 366 (e.g., a voltage, a temperature, a state of charge (SOC), a depth of discharge (DOD), a state of health (SOH), and/or a current of the battery 366).

More generally, the power driver circuit 370 can be configured to provide for (i) transmitting the electrosurgical energy from the first conductors 362 of the proximal cable 258 to the first subset of the second conductors 364 of the distal cable 260 and (ii) transmitting the DC power between the battery 366 and the second subset of the second conductors 364.

In one example, the power driver circuit 370 can include a power printed circuit board (PCB) 378 electrically coupled to the first set of contacts 372, the second set of contacts 374, and/or the third set of contacts 376. The power PCB 378 can include the electrical components for performing the power-related operations described above in connection with the battery 366 and/or providing the electrosurgical energy from the proximal cable 258 to the distal cable 260. In one example, the first set of contacts 372 can be at a proximal end of the power PCB 378, the second set of contacts 374 can be at a distal end of the power PCB 378, and the third set of contacts 376 can be positioned between the proximal end of the power PCB 378 and the distal end of the power PCB 378.

As shown in FIGS. 2-3, the plug 127 can include a plurality of prongs 280 that are configured to be received in respective sockets of the connector 120 of the electrosurgical generator 110. The prongs 280 can be provided in a quantity that corresponds to the quantity of the first conductors 362 of the proximal cable 258. In this arrangement, each prong 280 can be electrically coupled to a respective one of the first conductors 362 and provide for electrically coupling the respective one of the first conductors 362 to a respective one of the sockets of the connector 120. In the example shown in FIG. 2, the plug 127 three prongs 280 that are configured to couple to the electrosurgical generator 110. Accordingly, in the example of FIG. 2, the proximal cable 258 can include three first conductors 362, and the first contacts 372 can include three contacts. Also, in an implementation of the example shown in FIG. 2, the distal cable 260 can include five second contacts 374 and five second conductors 364 (e.g., including three second conductors 364 for the electrosurgical energy and two second conductors 364 for the DC power). As described above, other examples can include different quantities of the prongs 280, the first conductors 362, the first contacts 372, the second contacts 374, and/or the second conductors 364.

Referring now to FIGS. 4A-4F, an implementation of the battery module 142 is shown according to an example. In particular, FIG. 4A depicts a perspective view of the battery module 142 in an open state, FIG. 4B depicts a first side view of the battery module 142 in a closed state, FIG. 4C depicts a second side view of the battery module 142 in the closed state, FIG. 4D depicts a side view of the battery module 142 in the open state, FIG. 4E depicts a perspective view of the power driver circuit 370 shown in FIG. 4A, and FIG. 4F depicts a bottom view of the power driver circuit 370 shown in FIG. 4E, according to the example.

As shown in FIG. 4A, the battery module 142 includes the casing 368, which defines an internal compartment 482 that is configured to receive the battery 366. In example shown in FIGS. 4A-4H, the casing 368 is configured to receive a single battery 366. However, as described above, the battery 366 can include a plurality of batteries in other examples.

As described above, the battery module 142 can also include the power driver circuit 370 in the internal compartment 482 of the casing 368. As shown in FIGS. 4E-4F, the power driver circuit 370 can include (i) the first set of contacts 372 that are electrically coupled to the first conductors 362, (ii) the second set of contacts 374 that are electrically coupled to the second conductors 364, and (iii) the third set of contacts 376 that are electrically coupled to the battery 366. Additionally, as shown in FIGS. 4E-4F, the power driver circuit 370 can include a power printed circuit board (PCB) 378 electrically coupled to the first set of contacts 372, the second set of contacts 374, and/or the third set of contacts 376.

In this example, the first set of contacts 372 can be at a proximal end 478A of the power PCB 378, the second set of contacts 374 can be at a distal end 478B of the power PCB 378, and the third set of contacts 376 can be positioned between the proximal end 478A of the power PCB 378 and the distal end 478B of the power PCB 378. This can help to reduce the respective lengths of the proximal cable 258 and the distal cable 260, and/or help improve safety by placing relatively higher energy connections of the first set of contacts 372 and the second set of contacts 374 away from the third set of contacts 376, which the user may interact with while changing the battery 366.

As shown in FIGS. 4A and 4E, the battery module 142 can include one or more battery retention clips 484 positioned between respective ones of the third set of contacts 376. In this arrangement, the battery retention clip(s) 484 can assist in maintaining the battery 366 in a position in which the battery 366 electrically contacts the set of third contacts 376. Additionally, the one or more battery retention clips 484 can help to inhibit the battery 366 from inadvertently falling out of the battery module 142 when the casing 368 is in the open configuration. Although the one or more battery retention clip(s) 484 can provide several benefits, the battery module 142 can omit the battery retention clip(s) 484 in other examples.

As described above, in some examples, the battery 366 can be non-replaceable and the casing 368 can only have a closed that that inhibits access to the battery 366 in the internal compartment 482. However, in the implementation shown in FIGS. 4A-4H, the casing 368 can be actuated between (i) the closed state in which the casing 368 inhibits or prevents access to the battery 366 in the internal compartment 482, and (ii) the open state in which the casing 368 permits access to the battery 366 in the internal compartment 482 (e.g., the casing 368 can be opened and closed multiple times to facilitate inserting, removing, and/or replacing the battery 366).

For example, in FIGS. 4A-4H, to facilitate actuating the casing 368 between the open state and the closed state, the casing 368 includes a plurality of sections 468A-468C that are hingedly coupled to each other. The sections 468A-468C are configured to move between the open state (shown in FIGS. 4A and 4D) that provides access to the internal compartment 482 of the casing 368 and the closed state (shown in FIGS. 4B and 4C) that inhibits access to the internal compartment 482 of the casing 368.

The sections 468A-468C can include a bottom section 468A coupled to the power driver circuit 370, and a top section 468B hingedly coupled to the bottom section 468A. When the casing 368 is in the closed state, the bottom section 468A and the top section 468B can define the internal compartment 482. To secure the casing 368 in the closed state, the casing 368 can include a locking mechanism 486. As an example, in FIG. 4A-4H, the locking mechanism 486 includes a latch 486A (shown in FIGS. 4A and 4C) on the top section 468B that can engage a detent 486B (shown in FIG. 4B) on the bottom section 468A to releasably retain the casing in the closed state. In other examples, the locking mechanism 486 can additionally or alternatively include at least one of a group consisting of: a sliding latch, a threaded connection, a mechanical fastener (e.g., a screw), a spring latch, and a lock and key mechanism.

As shown in FIGS. 4A and 4D, the sections 468A-468C of the casing 368 can also include a middle section 468C that is hingedly coupled to the bottom section 468A. In this example, the casing 368 can transition from the open state to the closed state by first folding the middle section 468C over the bottom section 468A, and then folding the top section 468B over both the bottom section 468A and the middle section 468C. After folding the top section 468B over both the bottom section 468A and the middle section 468C, the bottom section 468A and the top section 468B can be secured to each other by the locking mechanism 486.

As shown in FIGS. 4A and 4D, the middle section 468C can include a cradle portion 488 that can be configured to receive the battery 366 when the battery 366 is coupled to the third contacts 376 and/or the battery retention clips 484. For example, the cradle portion 488 can have a concave shape that generally corresponds to outer surface profile of the battery 366. This can further help to retain the battery 366 in the position in which the battery 366 electrically contacts the set of third contacts 376.

As shown in FIG. 4A, the middle section 468C can include a plurality of apertures 490 that are each aligned with a respective one of the battery retention clips 484 and/or the third contacts 376. This can allow the battery retention clips 484 and/or the third contacts 376 to extend through the apertures 490 when the middle section 468C is positioned over the bottom section 468A. Additionally, to assist in retaining the battery 366 in the position in which the battery electrically contacts the third set of contacts 376, the top section 468B can include one or more ribs 392. Accordingly, in the arrangement shown in FIG. 4A, the battery 366 can be securely held in the position electrically contacting the set of third contacts 376 by (i) the battery retention clips 484, (ii) the cradle portion 488 of the middle section 468C, and (iii) the ribs 492 of the top section 468B.

As described above and shown in FIGS. 4A-4H, the proximal cable 258 and the distal cable 260 are each coupled to the power driver circuit 370. One challenge associated with this arrangement of the electrical cable 122 in which the proximal cable 258 is not integral or monolithic with the distal cable 260 is mitigating inadvertent decoupling between the power driver circuit 370 and the proximal cable 258, and/or mitigating inadvertent decoupling between the power driver circuit 370 and the distal cable 260. Within examples, the battery module 142 can include one or more features that can assist in coupling the proximal cable 258 and/or the distal cable 260 to the casing 368 and/or the power driver circuit 370 and thereby address such challenges of inadvertent decoupling.

For example, the battery module 142 can include a first clamp 494 at a proximal end of the casing 368, wherein the first clamp 494 is configured to apply a clamping force to the proximal cable 258, and a second clamp 496 at a distal end of the casing 368, wherein the second clamp 496 is configured to apply a clamping force to the distal cable 260. The clamping forces can help to mitigate against forces pulling the proximal cable 258 and/or the distal cable 260 away from the power driver circuit 370 and/or the casing 368.

As an example, in FIGS. 4A and 4G, the first clamp 494 can include a first recess 494A in the bottom section 468A and a first recess 494B in the top section 468B, the second clamp 496 can include a second recess 496A in the bottom section 468A and a second recess 496B in the top section 468B. The first clamp 494 can also include a first recess 494C in the middle section 468C, and the second clamp 496 can further include a second recess 496C in the middle section 468C. As noted above, the middle section 468C is positioned between the top section 468B and the bottom section 468A when the plurality of sections 468A-468C are in the closed state. Although the first clamp 494 can be defined by first recess 494A, the first recess 494B, or the first recess 494C in FIG. 4A, the first clamp 494 can be defined by two features selected from a group consisting of: the first recess 494A, the first recess 494B, and the first recess 494C in another example. Similarly, although the second clamp 496 can be defined by first recess 494A, the first recess 494B, or the first recess 494C in FIG. 4A, the second clamp 496 can be defined by two features selected from a group consisting of: the second recess 496A, the second recess 496B, or the second recess 496C in another example.

In this arrangement, when the casing 368 is in the closed state, the first clamp 494 can have a circumference that is less than a circumference of the proximal cable 258 to apply the clamping force to the proximal cable 258. Similarly, when the casing 368 is in the closed state, the second clamp 496 can have a circumference that is less than a circumference of the distal cable 260 to apply the clamping force to the distal cable 260.

In some examples, the battery module 142 can additionally or alternatively include a first strain relief structure 497 to assist in reducing strain on the proximal cable 258 and/or a second strain relief structure 498 to assist in reducing strain on the distal cable 260. As an example, in FIG. 4A, the casing 368 includes the first strain relief structure 497 at a position in the internal compartment 482 between the first clamp 494 and the power driver circuit 370, and the second strain relief structure 498 at a position in the internal compartment 482 between the power driver circuit 370 and the second clamp 496. In this example, the first strain relief structure 497 can include a first receptacle that is configured to receive the proximal cable 258, and the second strain relief structure 498 can include a second receptacle that is configured to receive the distal cable 260. The casing 368 has a length that extends in dimension between the proximal end of the casing 368 and the distal end of the casing 368. In a dimension that is perpendicular to the length, at least one of: (i) the first strain relief structure 497 is offset from the first clamp 494 and the first set of contacts 372 of the power driver circuit 370, or (ii) the second strain relief structure 498 is offset from the second clamp 496 and the second set of contacts 374 of the power driver circuit 370. Offsetting the first strain relief structure 497 and/or the second strain relief structure 498 can provide for the proximal cable 258 and/or the distal cable 260, respectively, to have a serpentine arrangement that reduces strain on the proximal cable 258 and/or the distal cable 260.

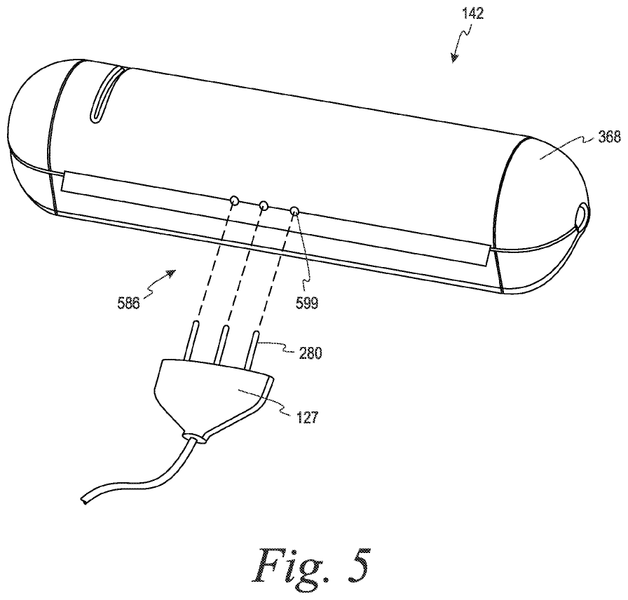

Referring now to FIG. 5, the battery module 142 is shown according to another example. The battery module 142 shown in FIG. 5 is substantially similar or identical to the battery module 142 shown in FIG. 1-4F except the battery module 142 shown in FIG. 5 includes a locking mechanism 586 that differs from the locking mechanism 468 of the battery module 142 shown in FIGS. 4A-4D.

In FIG. 5, the battery module 142 includes the locking mechanism 586 is configured to be actuated between a locked state and an unlocked state by a portion of the plug 127. In the locked state, the locking mechanism 586 inhibits or prevents the battery module 142 actuating from the closed state to the open state. In the unlocked state, the locking mechanism 586 allows the battery module 142 to actuate from the closed state to the open state. The locking mechanism 586 can help to mitigate inadvertently opening the casing 368 of the battery module 142.

In the example shown in FIG. 5, the locking mechanism 586 includes a keyhole 599 on the casing 368, and the keyhole 599 is configured to receive at least one of the prongs 280 of the plug 127 (e.g., three prongs 280 of the plug 127 in the example shown in FIG. 5). In this arrangement, the prong(s) 280 of the plug 127 can be inserted in the keyhole 599 to actuate the locking mechanism 586 from the locked state to the unlocked state. Configuring the locking mechanism 586 to be actuated by a portion of the plug 127 (e.g., one or more of the prongs 280) can mitigate the need for a separate key, thus ensuring that the mechanism for opening the battery module 142 is always at the same location as the battery module 142. However, in other examples, the locking mechanism 586 can additionally or alternatively include a separate key for actuating the locking mechanism 586.

In some examples, the locking mechanism 586 can be configured to automatically actuate from the unlocked state to the locked state responsive to actuating the battery module 142 from the open state to the closed state (e.g., without using a key). This can simplify operation of the locking mechanism 586. In other examples, the locking mechanism 586 can be configured such that the portion of the plug 127 and/or the separate key used to actuate the locking mechanism 586 from the closed state to the open state is also used to actuate the locking mechanism 586 from the open state to the closed state.

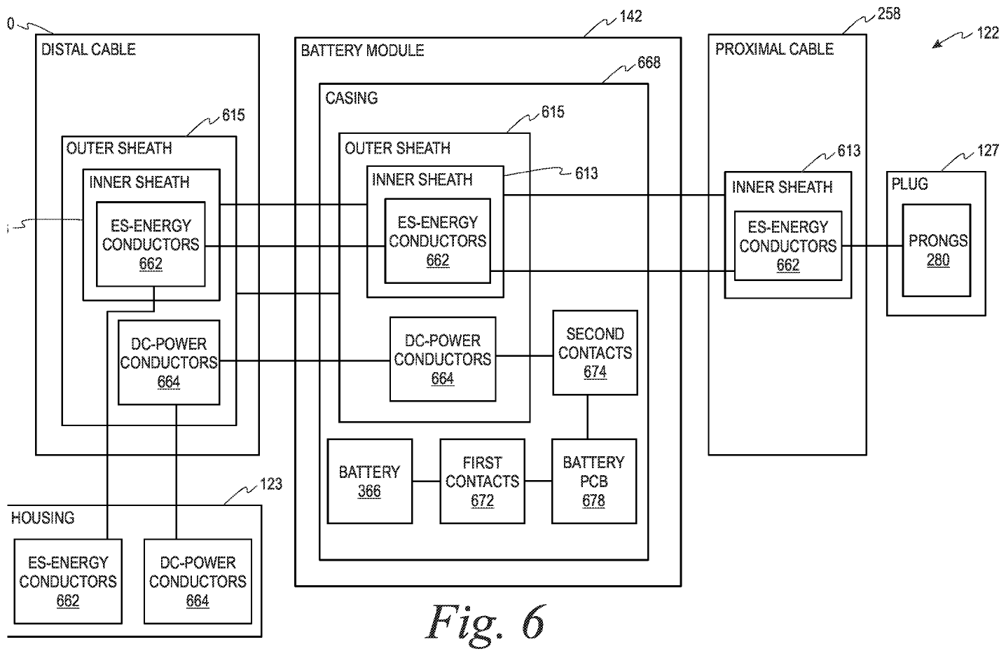

Referring now to FIG. 6, a simplified block diagram of the electrical cable 122 for the electrosurgical system 100 of FIG. 1, according to another example. As shown in FIG. 6, the electrical cable 122 includes the plug 127 that is configured to electrically couple to the electrosurgical generator 110, the proximal cable 258 extending from the plug 127 to the battery module 142, and the distal cable 260 extending from the battery module 142 to the housing 123. The battery module 142 includes a casing 668. The casing 368 defines an internal compartment that is configured to receive the battery 366. In some examples, the battery 366 can include a single battery. In other examples, the battery 366 can include a plurality of batteries. In some implementations, the casing 668 can completely enclose the battery 366. This can help to mitigate foreign matter (e.g., liquids) from contacting and affecting the operation of the battery 366. In other implementations, the casing 668 can retain the battery 366 without fully enclosing the battery 366.

In some examples, the battery 366 can be insertable and removable from the casing 368 (e.g., to facilitate inserting the battery 366 prior to first use of the electrosurgical tool 112 and/or to replace an expired battery with a new battery). In such examples, the casing 668 can be actuated between (i) a closed state in which the casing 668 inhibits or prevents access to the battery 366 in the internal compartment, and (ii) an open state in which the casing 668 permits access to the battery 366 in the internal compartment. In other examples, the battery 366 can be non-replaceable and the casing 668 can be configured to only inhibit access to the battery 366 in the internal compartment.

The battery module 142 can also include a battery PCB 678 in the internal compartment of the casing 668. The battery PCB 678 can include a plurality of first contacts 672 that electrically couple the battery 366 to the battery PCB 678. In some examples, the battery PCB 678 can include the electrical components for performing the power-related operations described above in connection with the battery 366 (e.g., providing the DC power from the battery 366 to the DC device 140). For instance, the battery PCB 678 can include one or more electrical components that can provide for reverse polarity protection, surge protection, electromagnetic interference (EMI) protection, overcharging protection, over-discharging protection, and/or over-drain protection in connection with the battery 366. The battery PCB 678 can additionally or alternatively provide a battery management system that can monitor a state of the battery 366 (e.g., a voltage, a temperature, a state of charge (SOC), a depth of discharge (DOD), a state of health (SOH), and/or a current of the battery 366).

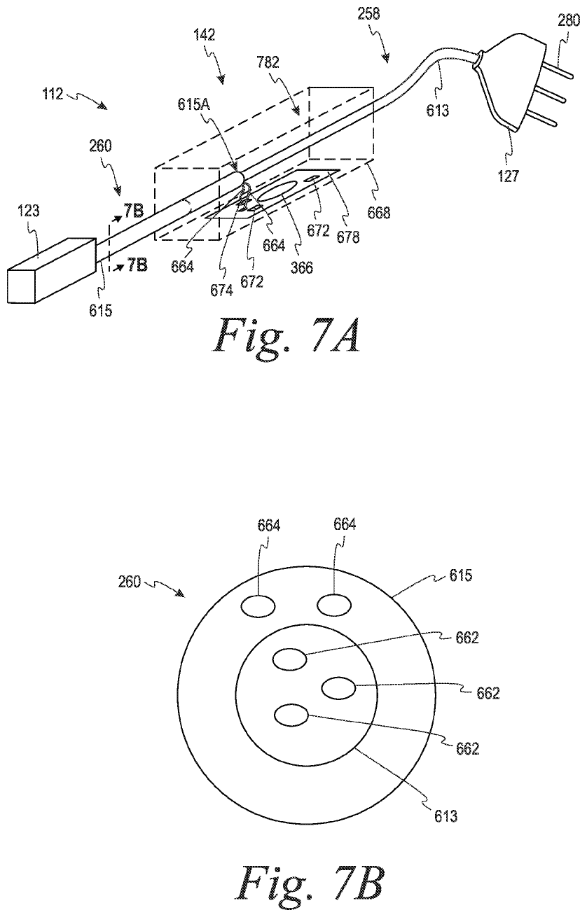

As shown in FIG. 6, the electrical cable 122 includes a plurality of electrosurgical energy (ES-energy) conductors 662 that extend an entire length of the electrical cable 122 between the plug 127 and the housing 123. The ES-energy conductors 662 can supply the electrosurgical energy from the electrosurgical generator 110 toward the electrosurgical electrode 128 (e.g., via the tool PCB 132, the housing conductor(s) 134, the shaft conductor(s) 136), and/or the electrosurgical electrode 128). As such, the ES-energy conductors 662 can extend from the prongs 280 of the plug 127 through the proximal cable 258, the casing 668 of the battery module 142, and the distal cable 260 to the housing 123. For instance, as shown in FIG. 6, the ES-energy conductors 662 can continuously extend (e.g., without interruption) through an inner sheath 613, and the inner sheath 613 can also extend from the plug 127 to the housing 123. In this arrangement, as shown and described below with respect to FIG. 7A, the ES-energy conductors 662 and the inner sheath 613 can extend through the internal compartment of the casing 368.

Additionally, as shown in FIG. 6, the electrical cable 122 includes a plurality of direct current power (DC-power) conductors 664 that extend from the battery PCB 678 to the housing 123. The DC-power conductors 664 can supply the DC power from the battery 366 toward the DC device 140. As such, the DC-power conductors 664 can extend from the battery module 142 through the distal cable 260 to the housing 123. For instance, as shown in FIG. 6, the DC-power conductors 664 can continuously extend (e.g., without interruption) through an outer sheath 615, and the outer sheath 615 can also extend from the casing 668 of the battery module 142 to the housing 123. As also shown in FIG. 6, the inner sheath 613 and the ES-energy conductors can also extend through the outer sheath 615 from the casing 668 of the battery module 142 to the housing 123. In FIG. 6, the battery PCB 678 includes a plurality of second contacts 674 that are configured to couple to the DC-power conductors 664.

The inner sheath 613 can be configured to bundle the ES-energy conductors 662, mitigate electromagnetic interference, and/or mitigate short circuit conditions (e.g., due to liquid ingress). Similarly, the outer sheath 615 can be configured to bundle the DC-power conductors 664 and the ES-energy conductors 662, mitigate electromagnetic interference, and/or mitigate short circuit conditions (e.g., due to liquid ingress). Further, in the arrangement shown in FIG. 6, the DC-power conductors 664 are separated from the ES-energy conductors 662 by the inner sheath 613. As such, the inner sheath 613 can help to mitigate electromagnetic interference and/or electrical noise due between the ES-energy conductors 662 and the DC-power conductors 664.

FIG. 7A depicts an implementation of the electrical cable 122 shown in FIG. 6, according to an example. FIG. 7B depicts a cross-sectional view of the electrical cable 122 taken through the distal cable 260. As shown in FIG. 7A, the electrical cable 122 includes the plug 127, the proximal cable 258, the battery module 142, and the distal cable 260. The proximal cable 258 extends from the plug 127 to the casing 668 of the battery module 142, and the distal cable 260 extends from the battery module 142 to the housing 123. The proximal cable 258 includes the inner sheath 613 in which the ES-energy conductors 662 are disposed, and distal cable 260 includes the outer sheath 615 in which the DC-power conductors 664, the inner sheath 613, and the ES-energy conductors 662 are disposed. As shown in FIG. 7A, the battery 366 is coupled to the battery PCB 678 by the first contacts 672, and the DC-power conductors 664 are coupled to the battery PCB 678 by the second contacts 674.

Additionally, in FIG. 7A, the inner sheath 613 (which encloses the ES-energy conductors 662) extends from the plug 127 along the proximal cable 258, entirely through the casing 668, and along the distal cable 260. As shown in FIG. 7B, a proximal end 615A of the outer sheath 615 can include an opening through which the inner sheath 613, the ES-energy conductors 662, and the DC-power conductors 664 can enter the outer sheath 615. The proximal end 615A of the outer sheath 615 can be disposed in an internal compartment 782 defined by the casing 668. This can help to reduce or prevent a risk of liquid entering the outer sheath 615 through the proximal end 615A.

As shown in FIG. 7B, at the distal cable 260, the ES-energy conductors 662 extend within the inner sheath 613, the inner sheath 613 extends within the outer sheath 615, and the DC-power conductors 664 extend within the outer sheath 615 and outside of the inner sheath 613.

As shown in FIGS. 6-7A, the plug 127 can include a plurality of prongs 280 that are configured to be received in respective sockets of the connector 120 of the electrosurgical generator 110, as described above. The prongs 280 can be provided in a quantity that corresponds to the quantity of the ES-energy conductors 662. In this arrangement, each prong 280 can be electrically coupled to a respective one of the ES-energy conductors 662 and provide for electrically coupling the respective one of the ES-energy conductors 662 to a respective one of the sockets of the connector 120. In the example shown in FIG. 7A, the plug 127 three prongs 280 that are configured to couple to the electrosurgical generator 110. Accordingly, in the example of FIGS. 7A-7B, the proximal cable 258 can include three ES-energy conductors 662. Also, in an implementation of the example shown in FIGS. 7A-7B, the distal cable 260 can include the ES-energy conductors 662 for the electrosurgical energy and two DC-power conductors 664 for the DC power. As described above, other examples can include different quantities of the prongs 280, the ES-energy conductors 662, and/or the DC-power conductors 664.

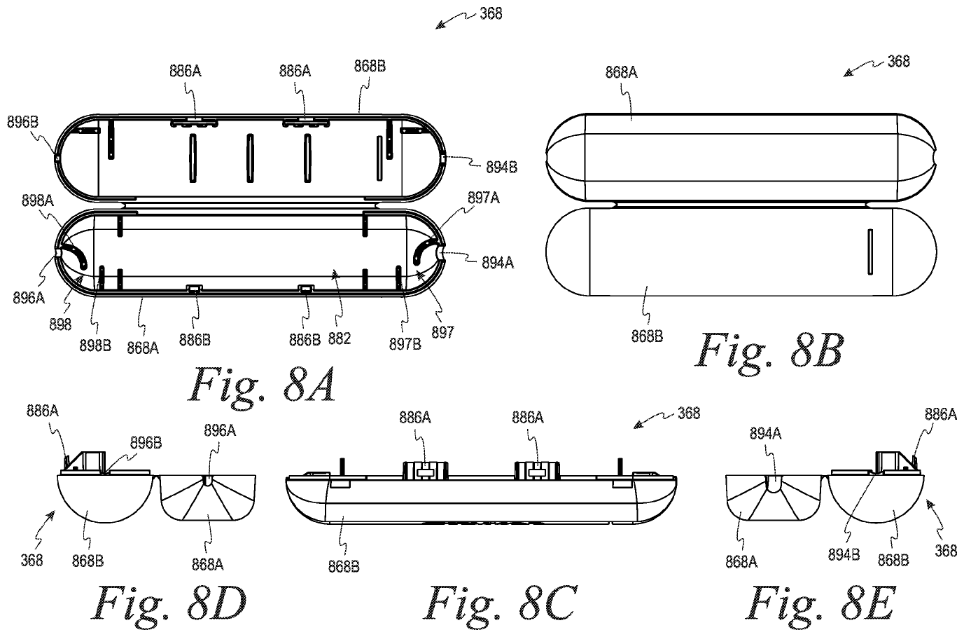

FIGS. 8A-8E shows another implementation of the casing 368 of the battery module 142 that can be used with either the electrical cable 122 shown in FIG. 3 and/or FIGS. 6-7B, according to another example. In particular, FIG. 8A depicts a top view of an internal compartment 882 of the casing 368 in an open state, FIG. 8B depicts a bottom view of an exterior surface of the casing 368 in the open state, FIG. 8C depicts a first side view of the casing 368 in the open state, FIG. 8D depicts a side view of a distal end of the casing 368 in the open state, and FIG. 8E depicts a side view of a proximal end of the casing 368 in the open state.

As shown in FIGS. 8A-8E, the casing 368, which defines the internal compartment 882 that is configured to receive the battery 366. In example shown in FIGS. 8A-8E, the casing 368 is configured to receive a single battery 366. However, as described above, the battery 366 can include a plurality of batteries in other examples.

Within examples, the casing 368 can be actuated from the open state shown in FIGS. 8A-8B to a closed state. As described above, when the casing 368 is in the open state, the casing 368 permits access to the internal compartment 882 and, when the casing 368 is in the closed state, the casing 368 can inhibit or prevent access to the internal compartment 882. In some examples, the casing 368 can be configured to be actuated between the closed state and the open state a plurality of times to facilitate inserting, removing, and/or replacing the battery 366 in the internal compartment 882 of the casing 368. However, in other examples including the example shown in FIGS. 8A-8B, the battery 366 can be non-replaceable and the casing 368 can be configured such that the casing 368 can be actuated from the open state to a closed state, but cannot be returned from the closed state to the open state. In this way, the casing 368 inhibits access to the battery 366 in the internal compartment 482.

To facilitate actuating the casing 368 between the open state and the closed state, the casing 368 can include a plurality of sections 868A, 868B that are moveable relative to each other. For example, in FIGS. 8A-8F, the casing 368 includes a first section 868A and a second section 868B that are hingedly coupled to each other. When the casing 368 is in the closed state, the first section 868A and the second section 868B can define the internal compartment 882. To secure the casing 368 in the closed state, the casing 368 can include a locking mechanism. As an example, in FIGS. 8A-8E, the locking mechanism includes one or more latches 886A on second section 868B that can engage a respective detent(s) 886B on the first section 868A to permanently retain the casing 368 in the closed state In other examples, the locking mechanism can additionally or alternatively include at least one of a group consisting of: a sliding latch, a threaded connection, a mechanical fastener (e.g., a screw), a spring latch, and a lock and key mechanism. As noted above, in other examples, the locking mechanism can alternatively be configured to provide a releasable coupling between the first section 868A and the second section 868B.

As described above, the battery module 142 can also include the power driver circuit 370 in the internal compartment 882 of the casing 368 for an implementation of the battery module 142 shown in FIG. 3, or the battery module 142 can include the battery PCB 678 for an implementation of the battery module 142 shown in FIG. 6. The power driver circuit 370 and/or the battery PCB 678 can be fixedly coupled to the first section 868A and/or the second section 868B of the casing 368. In the example shown in FIGS. 8A-8E, the first section 868A can have a generally planar surface that can assist in mounting the battery PCB 678 or the power driver circuit 370 to the casing 368. By contrast, the second section 868B can have a rounded contour, which can help to support a cylinder shaped battery in the internal compartment 882. However, in other examples, the first section 868A and/or the second section 868B can have different shapes, and/or the battery PCB 678 or the power driver circuit 370 can be coupled to the second section 868B.

Although not shown in FIGS. 8A-8E, the battery module 142 can additionally include one or more battery retention clips (e.g., the battery retention clip(s) 484 shown in FIGS. 4A and 4E) coupled to the first section 868A and/or the second section 868B. The one or more battery retention clips can help to inhibit the battery 366 from inadvertently falling out of the battery module 142 when the casing 368 is in the open configuration. Although the one or more battery retention clip(s) can provide several benefits, the battery module 142 can omit the battery retention clip(s) in other examples.

As shown in FIGS. 8D-8E, the casing 368 can include a first aperture at a proximal end of the casing 368 and a second aperture at a distal end of the casing 368. The first aperture is configured to receive the proximal cable 258 such that the proximal cable 258 extends through the first aperture into the interior compartment 882 of the casing 368, and the second aperture is configured to receive the distal cable 260 such that the distal cable extends through the second aperture into the interior compartment 882 of the casing 368

For example, as shown in FIG. 8E, the first section 868A can include a first recess 894A at the proximal end of the casing 368 and the second section 868B can include a second recess 894B at the proximal end of the casing 368. When the casing 368 is in the closed state, the first recess 894A and the second recess 894B are aligned with each other and define the first aperture through the proximal end of the casing 368. Additionally, the first recess 894A and the second recess 894B can be configured to apply a clamping force to the proximal cable 258 to help mitigate against forces pulling the proximal cable 258 outwardly away from the casing 368.

For example, as shown in FIG. 8D, the second section 868B can include a third recess 896A at the distal end of the casing 368 and the second section 868B can include a fourth recess 896B at the distal end of the casing 368. When the casing 368 is in the closed state, the third recess 896A and the fourth recess 896B are aligned with each other and define the second aperture through the distal end of the casing 368. Additionally, the third recess 896A and the fourth recess 896B can be configured to apply a clamping force to the distal cable 260 to help mitigate against forces pulling the distal cable 260 outwardly away from the casing 368

In this arrangement, when the casing 368 is in the closed state, the first aperture of the casing 368 (which is defined by the first recess 894A and the second recess 894B) can have a circumference that is less than a circumference of the proximal cable 258 to apply the clamping force to the proximal cable 258. Similarly, when the casing 368 is in the closed state, the second aperture of the casing 368 (which is defined by the third recess 896C and the fourth recess 896B) can have a circumference that is less than a circumference of the distal cable 260 to apply the clamping force to the distal cable 260.

In some examples, the casing 368 can additionally or alternatively include a first strain relief structure 897 to assist in reducing strain on the proximal cable 258 and/or a second strain relief structure 898 to assist in reducing strain on the distal cable 260. As an example, in FIG. 8A, the casing 368 includes the first strain relief structure 897 at a position in the internal compartment 882 adjacent to the first aperture at the proximal end of the casing 368, and the second strain relief structure 898 at a position in the internal compartment 482 adjacent to the second aperture at the distal end of the casing 368. For instance, in an implementation that includes the battery PCB 678, the first strain relief structure 897 can be between the battery PCB 678 and the first aperture at the proximal end of the casing 368, and the second strain relief structure 898 can be between the battery PCB 678 and the second aperture at the distal end of the casing 368. In an implementation that includes the power driver circuit 370, the first strain relief structure 897 can be between the power driver circuit 370 and the first aperture at the proximal end of the casing 368, and the second strain relief structure 898 can be between the power driver circuit 370 and the second aperture at the distal end of the casing 368

In the example shown in FIG. 8A, the first strain relief structure 897 can include one or more baffles that define a non-linear pathway for the proximal cable 258 through the internal compartment 882. For instance, in FIG. 8A, the first strain relief structure 897 includes a first baffle 897A that extends in a first direction that is transverse to a center axis of the first aperture in the proximal end of the casing 368. Additionally, the first strain relief structure 897 includes a second baffle 897B that is distal of the first baffle 897A and extends in a second direction that is transverse to the center axis of the first aperture. The first baffle 897A is configured to direct the proximal cable 258 in the first direction and the second baffle 897B is configured to direct the proximal cable 258 in the second direction such that the first strain relief structure 897 forms a bend in the proximal cable 258 passing through the first strain relief structure 897. By forming a bend around one or more baffles, the first strain relief structure 897 can help to reduce strain and/or reduce forces that may pull the proximal cable 258 outwardly away from the casing 368.

Additionally, in this example, the second strain relief structure 898 can include one or more baffles that define a non-linear pathway for the distal cable 260 in the internal compartment 882. For instance, in FIG. 8A, the second strain relief structure 898 includes a third baffle 898A that extends in a third direction that is transverse to a center axis of the second aperture in the proximal end of the casing 368. Additionally, the second strain relief structure 898 includes a fourth baffle 898B that is proximal of the third baffle 898A and extends in a fourth direction that is transverse to the center axis of the second aperture. The third baffle 898A is configured to direct the distal cable 260 in the third direction and the fourth baffle 898B is configured to direct the distal cable 260 in the fourth direction such that the second strain relief structure 898 forms a bend in the distal cable 260 passing through the second strain relief structure 898. By forming a bend around one or more baffles, the second strain relief structure 898 can help to reduce strain and/or reduce forces that may pull the distal cable 260 outwardly away from the casing 368

As shown in FIG. 8A, the first baffle 897A and the third baffle 898A extend from the proximal end and the distal end, respectively, to a point at which the first baffle 897A and the third baffle 898B are orthogonal to the first aperture and the second aperture, respectively. This can help to enhance the strain relief and cable retention provided by the first strain relief structure 897 and the second strain relief structure 898. Further, the first baffle 897A and the third baffle 898B can have a curved shape such that a gap is formed between the first baffle 897A and the proximal cable 258 and a gap is formed between the third baffle 898A and the distal cable 260. This can help to enhance the strain relief and cable retention provided by the first strain relief structure 897 and the second strain relief structure 898.

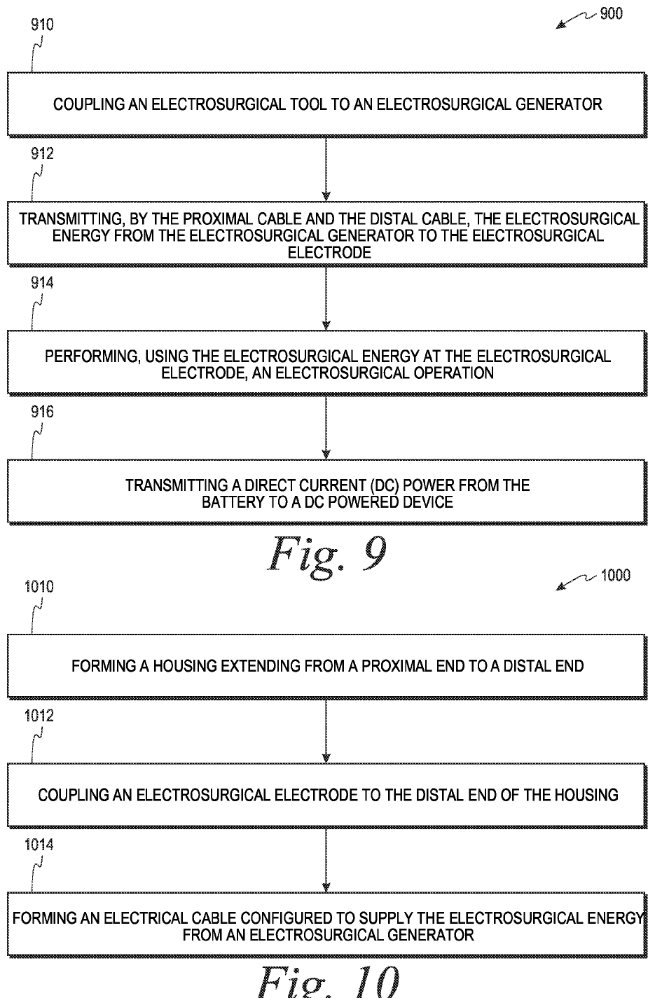

Referring now to FIG. 9, a flowchart of a process 900 for of operating an electrosurgical tool is shown according to an example. At block 910, the process 900 includes coupling an electrosurgical tool to an electrosurgical generator. The electrosurgical tool includes (i) a housing extending from a proximal end to a distal end, (ii) an electrosurgical electrode extending from the distal end of the housing, and (iii) an electrical cable extending from the proximal end of the housing. The electrical cable is configured to supply the electrosurgical energy from an electrosurgical generator. The electrosurgical electrode is configured to use electrosurgical energy to at least one of cut or coagulate tissue. The electrical cable includes: (a) a plug configured to electrically couple to an electrosurgical generator, (b) a proximal cable including a plurality of first conductors extending from the plug to a battery module, and (c) a distal cable including a plurality of second conductors extending from the battery module to the housing. The plurality of first conductors include a first quantity of conductors, the plurality of second conductors include a second quantity of conductors, and the first quantity is less than the second quantity.

The battery module includes a casing defining an internal compartment that is configured to receive a battery, and a power driver circuit in the internal compartment of the casing. The power driver circuit includes: (i) a first set of contacts that are electrically coupled to the plurality of first conductors, (ii) a second set of contacts that are electrically coupled to the plurality of second conductors, and (iii) a third set of contacts that are electrically coupled to the battery.

At block 912, the process 900 also includes transmitting, by the proximal cable and the distal cable, the electrosurgical energy from the electrosurgical generator to the electrosurgical electrode. At block 914, the process 900 includes performing, using the electrosurgical energy at the electrosurgical electrode, an electrosurgical operation. At block 916, the process 900 includes transmitting a direct current (DC) power from the battery to a DC powered device.

Referring now to FIG. 10, a flowchart for a process 1000 of forming an electrosurgical tool is shown according to an example. At block 1010, the process 1000 includes forming a housing extending from a proximal end to a distal end. At block 1012, the process 1000 includes coupling an electrosurgical electrode to the distal end of the housing. The electrosurgical electrode is configured to use electrosurgical energy to at least one of cut or coagulate tissue. At block 1014, the process 1000 includes forming an electrical cable configured to supply the electrosurgical energy from an electrosurgical generator.

The electrical cable includes (I) a plug configured to electrically couple to an electrosurgical generator, (II) a proximal cable including a plurality of first conductors extending from the plug to a battery module, and (III) a distal cable including a plurality of second conductors extending from the battery module to the housing. The plurality of first conductors includes a first quantity of conductors, the plurality of second conductors includes a second quantity of conductors, and the first quantity is less than the second quantity.

The battery module includes (a) a casing defining an internal compartment that is configured to receive a battery and (b) a power driver circuit in the internal compartment of the casing. The power driver circuit includes: (i) a first set of contacts that are electrically coupled to the plurality of first conductors, (ii) a second set of contacts that are electrically coupled to the plurality of second conductors, and (iii) a third set of contacts that are electrically coupled to the battery.

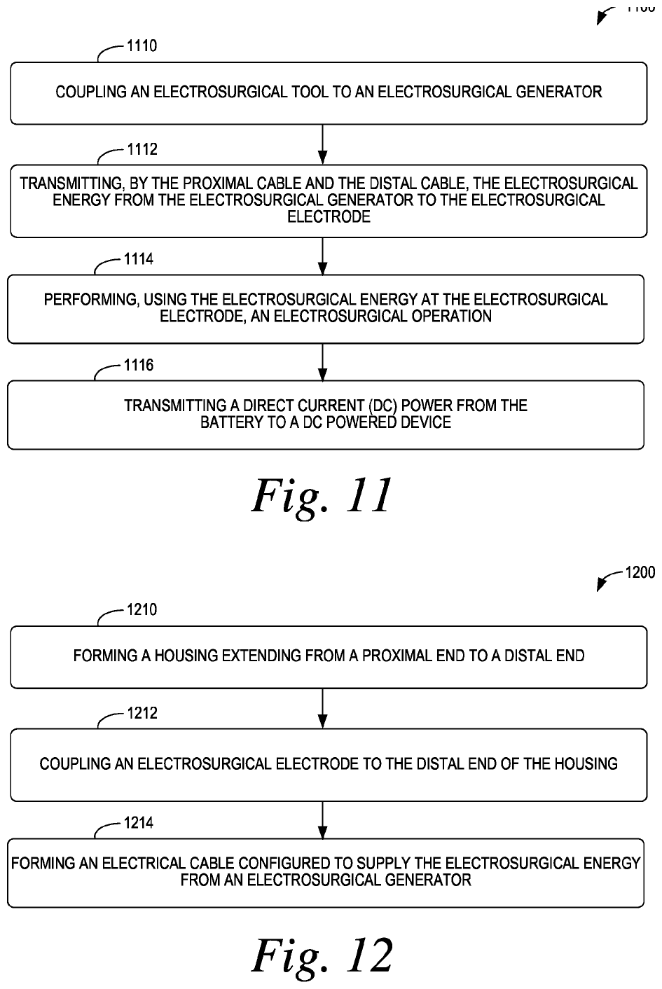

Referring now to FIG. 11, a flowchart of a process 1100 for of operating an electrosurgical tool is shown according to another example. At block 1110, the process 1100 includes coupling an electrosurgical tool to an electrosurgical generator. The electrosurgical tool includes (i) a housing extending from a proximal end to a distal end, (ii) an electrosurgical electrode extending from the distal end of the housing, and (iii) an electrical cable extending from the proximal end of the housing. The electrical cable is configured to supply the electrosurgical energy from an electrosurgical generator. The electrosurgical electrode is configured to use electrosurgical energy to at least one of cut or coagulate tissue.

The electrical cable includes a plug configured to electrically couple to the electrosurgical generator. The electrical cable also includes a battery module comprising: (i) a casing defining an internal compartment that is configured to receive a battery, and (ii) a battery PCB in the internal compartment of the casing. The battery PCB includes a first set of contacts that are configured to electrically couple a battery to the battery PCB. The electrical cable also includes a proximal cable extending from the plug to a battery module, a distal cable extending from the battery module to the housing, a plurality of ES-energy conductors extend an entire length of the electrical cable between the plug and the housing, and a plurality of DC-power conductors that extend from the battery PCB to the housing.

At block 1112, the process 1100 also includes transmitting, by the proximal cable and the distal cable, the electrosurgical energy from the electrosurgical generator to the electrosurgical electrode. At block 1114, the process 1100 includes performing, using the electrosurgical energy at the electrosurgical electrode, an electrosurgical operation. At block 1116, the process 900 includes transmitting a DC power from the battery to a DC powered device.

Referring now to FIG. 12, a flowchart for a process 1200 of forming an electrosurgical tool is shown according to an example. At block 1210, the process 1200 includes forming a housing extending from a proximal end to a distal end. At block 1212, the process 1200 includes coupling an electrosurgical electrode to the distal end of the housing. The electrosurgical electrode is configured to use electrosurgical energy to at least one of cut or coagulate tissue. At block 1214, the process 1200 includes forming an electrical cable configured to supply the electrosurgical energy from an electrosurgical generator

The electrical cable includes a plug configured to electrically couple to the electrosurgical generator. The electrical cable also includes a battery module comprising: (i) a casing defining an internal compartment that is configured to receive a battery, and (ii) a battery PCB in the internal compartment of the casing. The battery PCB includes a first set of contacts that are configured to electrically couple a battery to the battery PCB. The electrical cable also includes a proximal cable extending from the plug to a battery module, a distal cable extending from the battery module to the housing, a plurality of ES-energy conductors extend an entire length of the electrical cable between the plug and the housing, and a plurality of DC-power conductors that extend from the battery PCB to the housing.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs The breadth of the present application is not to be limited by the subject specification, but rather only by the plain meaning of the claim terms employed.

What is claimed is:

1. An electrosurgical tool, comprising:
a housing extending from a proximal end to a distal end;
an electrosurgical electrode extending from the distal end of the housing, wherein the electrosurgical electrode is configured to use electrosurgical energy to at least one of cut or coagulate tissue; and
an electrical cable extending from the proximal end of the housing, wherein the electrical cable is configured to supply the electrosurgical energy from an electrosurgical generator, wherein the electrical cable comprises:
a plug configured to electrically couple to the electrosurgical generator,
a proximal cable comprising a plurality of first conductors extending from the plug to a battery module,
a distal cable comprising a plurality of second conductors extending from the battery module to the housing,
wherein the plurality of first conductors comprises a first quantity of conductors, the plurality of second conductors comprises a second quantity of conductors, and the first quantity is less than the second quantity, and
wherein the battery module comprises:
a casing defining an internal compartment that is configured to receive a battery, and
a power driver circuit in the internal compartment of the casing, wherein the power driver circuit comprises: (i) a first set of contacts that are electrically coupled to the plurality of first conductors, (ii) a second set of contacts that are electrically coupled to the plurality of second conductors, and (iii) a third set of contacts that are electrically coupled to the battery.

2. The electrosurgical tool of claim 1, wherein the battery module comprises:
a first clamp at a proximal end of the casing, wherein the first clamp is configured to apply a clamping force to the proximal cable, and
a second clamp at a distal end of the casing, wherein the second clamp is configured to apply a clamping force to the distal cable.

3. The electrosurgical tool of claim 2, wherein the casing comprises:
a first strain relief structure at a position in the internal compartment between the first clamp and the power driver circuit, wherein the first strain relief structure comprises a first receptacle that is configured to receive the proximal cable; and
a second strain relief structure at a position in the internal compartment between the power driver circuit and the second clamp, wherein the second strain relief structure comprises a second receptacle that is configured to receive the distal cable.

4. The electrosurgical tool of claim 3, wherein the casing has a length that extends in dimension between the proximal end of the casing and the distal end of the casing, and
wherein, in a dimension that is perpendicular to the length, at least one of: (i) the first strain relief structure is offset from the first clamp and the first set of contacts of the power driver circuit, or (ii) the second strain relief structure is offset from the second clamp and the second set of contacts of the power driver circuit.

5. The electrosurgical tool of claim 2, wherein the casing comprises a plurality of sections that are hingedly coupled to each other, and
wherein the plurality of sections are configured to move between an open state that provides access to the internal compartment of the casing and a closed state that inhibits access to the internal compartment of the casing.

6. The electrosurgical tool of claim 5, wherein the plurality of sections comprises:
a bottom section coupled to the power driver circuit; and
a top section hingedly coupled to the bottom section,
wherein the first clamp comprises a first recess in the bottom section and a first recess in the top section, and
wherein the second clamp comprises a second recess in the bottom section and a second recess in the top section.

7. The electrosurgical tool of claim 6, wherein the plurality of sections further comprise a middle section that is hingedly coupled to the bottom section,
wherein the first clamp further comprises a first recess in the middle section, and
wherein the second clamp further comprises a second recess in the middle section, and
wherein the middle section is positioned between the top section and the bottom section when the plurality of sections are in the closed state.

8. The electrosurgical tool of claim 1, wherein the power driver circuit comprises a printed circuit board,
wherein first set of contacts are at a proximal end of the printed circuit board,
wherein the second set of contacts are at a distal end of the printed circuit board, and
wherein the third set of contacts are positioned between the proximal end of the printed circuit board and the distal end of the printed circuit board.

9. The electrosurgical tool of claim 8, further comprising one or more battery retention clips positioned between respective ones of the third set of contacts.

10. The electrosurgical tool of claim 1, wherein the first quantity of conductors is three and the second quantity of conductors is five.

11. The electrosurgical tool of claim 10, wherein the plurality of first conductors and a first subset of the plurality of second conductors are configured to supply the electrosurgical energy from the electrosurgical generator toward the electrosurgical electrode, and
wherein a second subset of the plurality of second conductors are configured to supply electrical power from the battery to a direct current (DC) device.

12. The electrosurgical tool of claim 11, further comprising the DC device, wherein the DC device comprises at least one device selected from a group consisting of: a light source, an ultrasound transmitter, a camera, one or more haptic devices, and one or more fluid pumps.

13. The electrosurgical tool of claim 10, wherein the plug comprises three prongs that are configured to couple to the electrosurgical generator.

14. The electrosurgical tool of claim 1, wherein the proximal cable has a first length, the distal cable has a second length, and the second length is greater than the first length.

15. A method of operating an electrosurgical tool, comprising:
coupling an electrosurgical tool to an electrosurgical generator, wherein the electrosurgical tool comprises:
a housing extending from a proximal end to a distal end,
an electrosurgical electrode extending from the distal end of the housing, wherein the electrosurgical electrode is configured to use electrosurgical energy to at least one of cut or coagulate tissue, and
an electrical cable extending from the proximal end of the housing, wherein the electrical cable is configured to supply the electrosurgical energy from the electrosurgical generator, wherein the electrical cable comprises: (a) a plug configured to electrically couple to the electrosurgical generator, (b) a proximal cable comprising a plurality of first conductors extending from the plug to a battery module, and (c) a distal cable comprising a plurality of second conductors extending from the battery module to the housing,
wherein the plurality of first conductors comprises a first quantity of conductors, the plurality of second conductors comprises a second quantity of conductors, and the first quantity is less than the second quantity, and
wherein the battery module comprises a casing defining an internal compartment that is configured to receive a battery, and a power driver circuit in the internal compartment of the casing, wherein the power driver circuit comprises: (i) a first set of contacts that are electrically coupled to the plurality of first conductors, (ii) a second set of contacts that are electrically coupled to the plurality of second conductors, and (iii) a third set of contacts that are electrically coupled to the battery;
transmitting, by the proximal cable and the distal cable, the electrosurgical energy from the electrosurgical generator to the electrosurgical electrode;
performing, using the electrosurgical energy at the electrosurgical electrode, an electrosurgical operation; and
transmitting a direct current (DC) power from the battery to a DC powered device.

16. The method of claim 15, further comprising:
actuating the casing from a closed state to an open state;
while the casing is in the open state, inserting the battery in the battery module; and
after inserting the battery in the battery module, actuating the casing from the open state to the closed state.

17. The method of claim 16, wherein the casing comprises a plurality of sections that are hingedly coupled to each other, and
wherein actuating the casing from the closed state to the open state comprises moving the plurality of sections relative to each other.

18. The method of claim 17, wherein the plurality of sections comprises:
a bottom section coupled to the power driver circuit; and
a top section hingedly coupled to the bottom section.

19. The method of claim 18, further comprising:
applying, by a first clamp at a proximal end of the casing, a clamping force to the proximal cable a first clamp at a proximal end of the casing; and
applying, by a second clamp at a distal end of the casing, a clamping force to the distal cable,
wherein the first clamp comprises a first recess in the bottom section and a first recess in the top section, and
wherein the second clamp comprises a second recess in the bottom section and a second recess in the top section.

20. The method of claim 15, wherein the first quantity of conductors is three and the second quantity of conductors is five.

21. The method of claim 19, wherein transmitting, by the proximal cable and the distal cable, the electrosurgical energy from the electrosurgical generator to the electrosurgical electrode comprises transmitting the electrosurgical electrode using the plurality of first conductors and a first subset of the plurality of second conductors.

22. The method of claim 20, transmitting the DC power from the battery to the DC device comprises transmitting the DC power using a second subset of the plurality of second conductors.

23. The method of claim 15, further comprising operating, using the DC power, the DC device, wherein the DC device comprises at least one device selected from a group consisting of: a light source, an ultrasound transmitter, a camera, one or more haptic devices, and one or more fluid pumps.

24. A method of forming an electrosurgical tool, comprising:
forming a housing extending from a proximal end to a distal end;
coupling an electrosurgical electrode to the distal end of the housing, wherein the electrosurgical electrode is configured to use electrosurgical energy to at least one of cut or coagulate tissue; and
forming an electrical cable configured to supply the electrosurgical energy from an electrosurgical generator, wherein the electrical cable comprises:
a plug configured to electrically couple to the electrosurgical generator,
a proximal cable comprising a plurality of first conductors extending from the plug to a battery module,
a distal cable comprising a plurality of second conductors extending from the battery module to the housing,
wherein the plurality of first conductors comprises a first quantity of conductors, the plurality of second conductors comprises a second quantity of conductors, and the first quantity is less than the second quantity, and
wherein the battery module comprises:
a casing defining an internal compartment that is configured to receive a battery, and
a power driver circuit in the internal compartment of the casing, wherein the power driver circuit comprises: (i) a first set of contacts that are electrically coupled to the plurality of first conductors, (ii) a second set of contacts that are electrically coupled to the plurality of second conductors, and (iii) a third set of contacts that are electrically coupled to the battery.

25. An electrosurgical tool, comprising:
a housing extending from a proximal end to a distal end;
an electrosurgical electrode extending from the distal end of the housing, wherein the electrosurgical electrode is configured to use electrosurgical energy to at least one of cut or coagulate tissue; and
an electrical cable extending from the proximal end of the housing, wherein the electrical cable is configured to supply the electrosurgical energy from an electrosurgical generator, wherein the electrical cable comprises:
a plug configured to electrically couple to the electrosurgical generator,
a battery module comprising: (i) a casing defining an internal compartment that is configured to receive a battery, and (ii) a battery printed circuit board (PCB) in the internal compartment of the casing, wherein the battery PCB comprises a first set of contacts that are configured to electrically couple a battery to the battery PCB,
a proximal cable extending from the plug to a battery module,
a distal cable extending from the battery module to the housing,
a plurality of electrosurgical energy (ES-energy) conductors extend an entire length of the electrical cable between the plug and the housing, and
a plurality of direct current power (DC-power) conductors that extend from the battery PCB to the housing.

26. The electrosurgical tool of claim 25, wherein the battery PCB comprises a plurality of second contacts that are configured to couple to the plurality of DC-power conductors.

27. The electrosurgical tool of any one of claims 25-26, wherein the electrical cable further comprises an inner sheath extending from the plug to the housing, wherein the plurality of ES-energy conductors continuously extend through the inner sheath.

28. The electrosurgical tool of claim 27, wherein the plurality of ES-energy conductors and the inner sheath can extend through the internal compartment of the casing.

29. The electrosurgical tool of any one of claims 27-28, wherein the electrical cable further comprises an outer sheath that extend from the casing of the battery module to the housing, wherein the plurality of DC-power conductors continuously extend through the outer sheath.

30. The electrosurgical tool of claim 29, wherein the inner sheath and the plurality of ES-energy conductors also extend through the outer sheath.

31. The electrosurgical tool of any one of claims 29-30, wherein a proximal end of the outer sheath comprises an opening through which the inner sheath, the plurality of ES-energy conductors, and the plurality of DC-power conductors enter the outer sheath, wherein the proximal end of the outer sheath is disposed in the internal compartment defined by the casing.

32. The electrosurgical tool of any one of claims 29-31, wherein the proximal cable comprises the plurality of ES-energy conductors and the inner sheath, and
wherein the distal cable comprises the plurality of ES-energy conductors, the inner sheath, the plurality of DC-power conductors, and the outer sheath.

33. The electrosurgical tool of any one of claims 25-32, wherein the casing comprises a first section and a second section that are hingedly coupled to each other, and
wherein the first section and the second section are configured to move between an open state that provides access to the internal compartment of the casing and a closed state that inhibits access to the internal compartment of the casing

34. The electrosurgical tool of claim 33, wherein the first section comprises a first recess at the proximal end of the casing and the second section comprises a second recess at the proximal end of the casing,
wherein, when the casing is in the closed state, the first recess and the second recess define a first aperture through the proximal end of the casing and are configured to apply a clamping force to the proximal cable,
wherein the second section comprises a third recess at the distal end of the casing and the second section comprises a fourth recess at the distal end of the casing, and
wherein, when the casing is in the closed state, the third recess and the fourth recess define a second aperture through the distal end of the casing and are configured to apply a clamping force to the distal cable.

35. The electrosurgical tool of claim 34, wherein the battery module further comprises a first strain relief structure at a position in the internal compartment adjacent to the first aperture at the proximal end of the casing, and the second strain relief structure at a position in the internal compartment adjacent to the second aperture at the distal end of the casing.

36. The electrosurgical tool of claim 35, wherein the first strain relief structure comprises one or more baffles that define a non-linear pathway for the proximal cable through the internal compartment, and the second strain relief structure comprises one or more baffles that define a non-linear pathway for the distal cable in the internal compartment.

37. The electrosurgical tool of claim 36, wherein the first strain relief structure comprises a first baffle that extends in a first direction that is transverse to a center axis of the first aperture in the proximal end of the casing, and a second baffle that is distal of the first baffle and extends in a second direction that is transverse to the center axis of the first aperture,
wherein the first baffle is configured to direct the proximal cable in the first direction and the second baffle is configured to direct the proximal cable in the second direction such that the first strain relief structure forms a bend in the proximal cable passing through the first strain relief structure.

38. The electrosurgical tool of any one of claims 36-37, wherein the second strain relief structure comprises a third baffle that extends in a third direction that is transverse to a center axis of the second aperture in the proximal end of the casing, and a fourth baffle that is proximal of the third baffle and extends in a fourth direction that is transverse to the center axis of the second aperture, and
wherein the third baffle is configured to direct the distal cable in the third direction and the fourth baffle is configured to direct the distal cable in the fourth direction such that the second strain relief structure forms a bend in the distal cable passing through the second strain relief structure.

39. The electrosurgical tool of any one of claims 25-38, wherein the plurality of ES-energy conductors comprises three ES-energy conductors, and wherein the plurality of DC-power conductors comprises two DC-power conductors.

40. The electrosurgical tool of claim 36, wherein the plug comprises three prongs that are configured to couple to the electrosurgical generator.

41. The electrosurgical tool of any one of claims 25-40, further comprising the DC device, wherein the DC device comprises at least one device selected from a group consisting of: a light source, an ultrasound transmitter, a camera, one or more haptic devices, and one or more fluid pumps.

42. The electrosurgical tool of any one of claims 25-41, wherein the proximal cable has a first length, the distal cable has a second length, and the second length is greater than the first length.

43. A method of operating an electrosurgical tool, comprising:
coupling an electrosurgical tool to an electrosurgical generator, wherein the electrosurgical tool comprises:
a housing extending from a proximal end to a distal end;
an electrosurgical electrode extending from the distal end of the housing, wherein the electrosurgical electrode is configured to use electrosurgical energy to at least one of cut or coagulate tissue; and
an electrical cable extending from the proximal end of the housing, wherein the electrical cable is configured to supply the electrosurgical energy from an electrosurgical generator, wherein the electrical cable comprises:
(a) a plug configured to electrically couple to the electrosurgical generator,
(b) a battery module comprising: (i) a casing defining an internal compartment that is configured to receive a battery, and (ii) a battery printed circuit board (PCB) in the internal compartment of the casing, wherein the battery PCB comprises a first set of contacts that are configured to electrically couple a battery to the battery PCB,
(c) a proximal cable extending from the plug to a battery module,
(d) a distal cable extending from the battery module to the housing,
(e) a plurality of electrosurgical energy (ES-energy) conductors extend an entire length of the electrical cable between the plug and the housing, and
(f) a plurality of direct current power (DC-power) conductors that extend from the battery PCB to the housing;
transmitting, by the proximal cable and the distal cable, the electrosurgical energy from the electrosurgical generator to the electrosurgical electrode;
performing, using the electrosurgical energy at the electrosurgical electrode, an electrosurgical operation; and
transmitting a direct current (DC) power from the battery to a DC powered device.

44. A method of forming an electrosurgical tool, comprising:
forming a housing extending from a proximal end to a distal end;
coupling an electrosurgical electrode to the distal end of the housing, wherein the electrosurgical electrode is configured to use electrosurgical energy to at least one of cut or coagulate tissue; and
forming an electrical cable configured to supply the electrosurgical energy from an electrosurgical generator, wherein the electrical cable comprises:
a plug configured to electrically couple to the electrosurgical generator,
a battery module comprising: (i) a casing defining an internal compartment that is configured to receive a battery, and (ii) a battery printed circuit board (PCB) in the internal compartment of the casing, wherein the battery PCB comprises a first set of contacts that are configured to electrically couple a battery to the battery PCB,
a proximal cable extending from the plug to a battery module,
a distal cable extending from the battery module to the housing,
a plurality of electrosurgical energy (ES-energy) conductors extend an entire length of the electrical cable between the plug and the housing, and
a plurality of direct current power (DC-power) conductors that extend from the battery PCB to the housing.
